(19) United States
(12) Patent Application Publication
(10) Pub. No.: US 2025/0205468 A1
(43) Pub. Date: Jun. 26, 2025

(54) SMART PROGRAMMABLE BLOOD PUMP FOR CONTROLLING AND AUTOMATING EXTRACORPOREAL LIFE SUPPORT APPLICATIONS

(71) Applicant: VANDERBILT UNIVERSITY, Nashville, TN (US)

(72) Inventors: Rei Ukita, Nashville, TN (US); Matthew Bacchetta, Nashville, TN (US); Gabriella Glomp, Nashville, TN (US); Michael Cortelli, Nashville, TN (US)

(21) Appl. No.: 18/989,242

(22) Filed: Dec. 20, 2024

Related U.S. Application Data
(60) Provisional application No. 63/613,091, filed on Dec. 21, 2023.

(51) Int. Cl. A61M 60/122

(52) U.S. Cl. CPC A61M 60/122

(57) ABSTRACT

One aspect of the invention provides a blood pump system comprising a blood pump configured to provide a blood flow in an extracorporeal membrane oxygenation (ECMO) circuit for extracorporeal physiological support for a patient; a magnetic coupling member configured to drive the blood pump to operably provide the blood flow; a direct current (DC) motor configured to drive the magnetic coupling member, thereby driving the blood pump; and a controller configured to regulate a motor speed of the DC motor.

### CROSS-REFERENCE TO RELATED PATENT APPLICATION

This application claims priority to and the benefit of U.S. Provisional Patent Application Ser. No. 63/613,091, filed Dec. 21, 2023, which is incorporated herein in its entirety by reference.

### FIELD OF THE INVENTION

The invention relates generally to blood pump systems, and more particularly, a smart programmable blood pump for controlling and automating extracorporeal life support applications.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Patients suffering from cardiopulmonary failure, difficult post-operative recoveries after transplant, severe trauma, and infection may need extracorporeal membrane oxygenation (ECMO) support. ECMO includes a mechanical blood pump and an oxygenator to provide extracorporeal physiologic support (FIG. 1). Patients can remain on ECMO from days to weeks as a bridge to recovery or to transplant. Between 2019 and 2024, there have been more than 102,000 ECMO runs, which makes up more than half of all reported cases since 1989 and demonstrates a rapid growth in the clinical demand for this technology. Within this period, ECMO was especially important for supporting COVID-19 patients. However, the high resource cost of therapy necessitates rigorous patient selection for ECMO, and this led to potentially preventable deaths during the peak of the COVID-19 pandemic. These limitations serve as a clinical motivation to innovate the enabling technology of ECMO to make the therapy less resource-intensive, particularly the labor cost. At an average hospital in the U.S., supporting a patient on ECMO costs $4,584 to $11,524 per day including materials, pharmaceuticals, machines, specialized personnel, procedures, and hospital stay. Personnel cost can be up to 80% of the total cost. Bedside staff must be vigilant in assessing the patient's condition and ensuring that the ECMO system adequately supports the patient. This process, as it exists, is labor-intensive and time-consuming, strains healthcare worker availability, and increases susceptibility to errors. Semi-automating any part of the manual responsibilities will help decrease the labor cost and expand the access of care, which is especially important in the settings of respiratory disease outbreak and mass casualty events.

To advance research and innovation in this area, we need to address the barriers that may be hindering progress. Namely, standard clinical machines for operating ECMO-especially the blood pump console—are expensive. Clinical centrifugal pump consoles can cost more than $40,000. Furthermore, the high level of information security built into the clinical systems limits the user's ability to program or integrate with a third-party device, which is crucial to implementing features such as automation. Even blood pump consoles solely for preclinical use can cost more than $10,000. A low-cost, programmable pump console would address this issue by making the technology more accessible.

Furthermore, a low-cost programmable pump console can also help advance research in the scientific area of pulsatility. Pulsatility in extracorporeal circulation has been a much-debated concept about its potential benefit over constant flow mode. There has been little research to prove the benefits of pulsatility in terms of improving end-organ perfusion, reducing clot burden inside the oxygenator, and improving gas transfer rates within the system and the body. Currently, the Novalung Heart & Lung system-specifically its DP3 diagonal pump—is the only clinically approved pump for extracorporeal applications that can provide pulsatile flow. Meanwhile, we have a limited understanding of how other clinical ECMO pumps function under a pulsatile setting.

Therefore, a heretofore unaddressed need exists in the art to address the aforementioned deficiencies and inadequacies.

### SUMMARY OF THE INVENTION

One of the objectives of this invention is to develop a low-cost, programmable console that can operate centrifugal blood pumps used in extracorporeal membrane oxygenation (ECMO).

In one aspect, the invention relates to a blood pump system comprising a blood pump configured to provide a blood flow in an extracorporeal membrane oxygenation (ECMO) circuit for extracorporeal physiological support for a patient; a magnetic coupling member configured to drive the blood pump to operably provide the blood flow; a direct current (DC) motor configured to drive the magnetic coupling member, thereby driving the blood pimp; and a controller configured to regulate a motor speed of the DC motor.

In one embodiment, the blood pump is a standard ECMO blood pump, or a customized blood pump.

In one embodiment, the blood flow is a constant blood flow, or a pulsatile blood flow.

In one embodiment, the magnetic coupling member is configured to rotate a pump impeller of the blood pump in a contactless manner.

In one embodiment, the blood pump system further comprises a Hall effect sensor positioned in relation to the magnetic coupling member to measure a pump speed of the blood pump in real time. The controller is further configured to receive a signal of the pump speed from the Hall effect sensor and regulate the motor speed of the DC motor responsively.

In one embodiment, the blood pump system further comprises a physiological sensor disposed in the ECMO circuit for monitor physiological parameters of the patient.

In one embodiment, the controller is further configured to receive a physiological input from the physiological sensor in the ECMO circuit and regulate the motor speed responsively, thereby adjusting the pump speed in a closed-loop feedback system.

In one embodiment, the controller is further configured to receive a signal from the patient about its vital state and regulate the motor speed responsively, thereby adjusting the pump speed.

In one embodiment, the controller is further configured to receive a user-scripted input to operate the blood pump system in a pulsatile or periodic manner.

In one embodiment, the blood pump system is programed to vary the motor speed in a constant manner or a pulsatile manner.

In one embodiment, the blood pump system is programed to follow a setpoint value to correct homeostasis.

In one embodiment, the blood pump system further comprises a feedback mechanism for controlling the pump speed by using venous saturation as a surrogate measure for patient status and basis for speed adjustment, so as to enable automation according to metabolic need of the patient.

In one embodiment, the feedback mechanism comprises a venous saturation monitor including an oxygen sensor coupled in the ECMO circuit to monitor mixed venous oxygen saturation (SvO₂) of the patient.

In one embodiment, the blood pump system is programmed, such that the pump speed is automatically adjusted to maintain a setpoint flow for physiologic oxygen saturation.

In one embodiment, the blood pump system further comprises a safety mechanism for ensuring the blood pump not to blindly increase speed in response to an abnormal ECMO circuit condition including circuit obstruction, low fluid volume status, clot formation, a suction event, pump thrombosis, and/or circuit thrombosis.

In one embodiment, the blood pump system is operable at clinically relevant levels of flow and pressure needed in extracorporeal life support applications.

In another aspect, the invention relates to a method of operating the blood pump system for extracorporeal life support applications. The method comprises driving the DC motor, thereby the blood pump at a target pump speed to provide the blood flow in the ECMO circuit for extracorporeal physiological support for the patient in an automatic mode or a manual mode, wherein the target pump speed is determined based on preset operation parameters.

In one embodiment, the preset operation parameters include setpoint flow and/or pressure in the ECMO circuit, motor current, oxygen saturation, and/or a physiological state of the patient.

In one embodiment, the physiological state of the patient includes an oxygen level, an oxygen consumption, and/or mixed venous oxygen saturation (SvO₂).

In one embodiment, the method further comprises measuring the pump speed of in real time; and regulating the motor speed responsively to adjust the pump speed when the measured pump speed is deviated from the target pump speed.

In one embodiment, the method further comprises measuring the SvO₂; and regulating the motor speed responsively to adjust the pump speed when the measured SvO₂ is deviated from a setpoint value.

In one embodiment, the method further comprises measuring the flow rate and/or pressure in the ECMO circuit; and regulating the motor speed responsively to adjust the pump speed when the measured flow rate and/or pressure are deviated from the setpoint flow and/or pressure.

In one embodiment, the method further comprises detecting an abnormal ECMO circuit condition including circuit obstruction, low fluid volume status, clot formation, a suction event, pump thrombosis, and/or circuit thrombosis; and regulating the motor speed responsively to ensure the blood pump not to blindly increase speed when the abnormal ECMO circuit condition is detected.

In one embodiment, when the abnormal ECMO circuit condition is detected, the blood pump system exits from the automatic mode and enters the manual mode until the abnormal ECMO circuit condition is resolved.

These and other aspects of the invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The invention may be better understood by reference to one or more of these figures in combination with the detailed description of specific embodiments presented herein. The drawings described below are for illustration purposes only, and are not intended to limit the scope of the invention in any way. Wherever possible, the same reference numbers are used throughout the drawings to refer to the same or like elements of an embodiment.

FIG. 1 is a diagram of veno-venous extracorporeal membrane oxygenation circuit. Created with BioRender.

FIG. 2 shows schematically a diagram of a smart blood pump system, according to embodiments of the invention.

FIG. 3 shows schematically a diagram for the smart blood pump incorporating circuit parameters to intelligently adjust the pump speed, according to embodiments of the invention.

FIG. 4 shows schematically a flow chart for pump speed adjustment decision process in the smart blood pump system, according to embodiments of the invention. The flowchart screens for potential common mechanical complications in the circuit including suction, cannula malposition, thrombosis, and circuit occlusion that can be troubleshooted using pressure, flow, and oxygen saturation signals.

FIG. 5 shows pump speed at varying voltages, and pressure-flow curves for varying levels of input voltage, according to embodiments of the invention. LivaNova Revolution was used as the pump head.

FIG. 6 shows sinusoidal rotational speed (top row, RPM ranging from 1100 to 1400 rpm) that is able to achieve pulsatile flow (4.8 to 5.8 L/min) and pressure head (23 to 41 mmHg) waveforms with a period of 1.5 seconds, with the Smart Blood Pump using the following settings: input voltage of 13.5 volts, Arduino variables minPWM, maxPWM, and period values of 180, 255, 1000 milliseconds, respectively, according to embodiments of the invention.

FIG. 7 shows a proportional-integral control scheme implemented for automation of the smart blood pump, according to embodiments of the invention. The pump titrates its rotational speed (top, revolutions per minute, RPM) to a setpoint flow rate of 3.5 L/min (dashed line, bottom). At the start, the pump was manually set at 1000 RPM at 2 L/min. As soon as the automatic mode is activated, the pump achieves the setpoint flow of 3.5 L/min within 5 seconds. The red triangle denotes acute occlusion of the circuit that induces a transient drop in the flow, and green triangles denote acute dilation of the circuit to induce a transient flow increase. The Smart Blood Pump responds by increasing or decreasing the speed to maintain the setpoint flow of 3.5 L/min.

FIG. 8 shows schematically an exemplary platform of the smart blood pump system for controlling the level of oxygen saturation in blood, according to embodiments of the invention. Oxygenation and deoxygenation circuits are run in parallel to titrate venous oxygen saturation (SvO₂). Once a target SvO₂ is set to simulate low or high SvO₂, the smart blood pump is activated to verify how quickly setpoint SvO₂ can be restored after its disturbances.

FIG. 9 shows schematically varying levels of mixed SvO₂ attained at different flow combinations between oxygenator and deoxygenator circuits in the circuit shown in FIG. 8.

FIG. 10 are representative time trajectory of oxygen saturation level, using the circuit setup described in FIG. G. Curve fitting yields a time constant of 60 seconds, which represents the amount of time taken to achieve 64% of the total change. This supports the invention rationale that the smart blood pump does not need to change the speed rapidly, as there is this time lag to consider before making any adjustments in the pump. The gradual speed changing approach will reduce overshooting and oscillation in the pump speed, which will also help reduce electrical current consumption, heating, and hemolysis.

FIG. 11 shows a photograph of the Smart Blood Pump. The enclosure was prototyped with laser-cut wooden pieces. A DC motor rated at 3000 RPM at 12 volts was used.

FIG. 12 shows two-dimensional computer-aided drawing of the enclosure panels for laser cutting. All annotated dimensions are in inches. For circular dimensions, R indicates the radius and Ø represents diameter. See the Zenodo repository for the actual file.

FIG. 13 shows assembly instructions for the enclosure. Color coding and numbers are provided to show how different panels should fit together. See the Zenodo repository for the actual file.

FIG. 14 shows three-dimensional rendering of (panel A) the assembled Smart Blood Pump enclosure, and (panel B) the exploded view of the enclosure.

FIG. 15 shows assembled prototyped enclosure with wood sheets with the panels labeled.

FIG. 16 shows a Hall effect sensor setup for measuring pump speed. Rpu=pull-up resistor.

FIG. 17 shows electrical connection for manual control of the Smart Blood Pump using a variable voltage power supply.

FIG. 18 shows screw terminals of the motor driver shield attached on top of the Sparkfun Redboard microcontroller are used for connecting to the input voltage source (green) and to the output voltage to the motor (black).

FIG. 19 shows bird's eye view of the programmable control setup of the Smart Blood Pump using the Sparkfun Redboard microcontroller and its Cytron motor driver shield.

FIG. 20 shows A) constructed flow circuit loop using the LivaNova Revolution pump, B) and the circuit primed with bovine whole blood.

FIG. 21 shows transonic flow meter setup and wiring guide to enable servo control of the flow rate with the Smart Blood Pump.

FIG. 22 shows data acquisition setup for monitoring flow, pressure, temperature, and pump speed during flow circuit loop testing.

FIG. 23 shows normalized Index of Hemolysis (NIH) from blood flow loop test utilizing Smart Pump platform with LivaNova Pump head. Data shown as mean+/−standard deviation (n=3). Horizontal line in plot below represents average from the study of Han et al.

FIG. 24 shows servo control of the flow rate is implemented in the Smart Blood Pump to maintain setpoint flow rate of (panel A) 2 L/min and (panel B) 3 L/min.

FIG. 25 shows oxygenation trajectory's exponential characteristic time constant, t (tau), is dependent on the circuit blood volume and flow rate. The ratio between blood volume and flow has a direct relationship with the exponential constant.

FIG. 26 shows pump automated control was tested with low and high resistance circuits, represented as blue and orange curves respectively, using the same control parameters (setpoint flow rate=3 L/min; gain coefficients of Kp=0.175 and Ki=1.0).

FIG. 27 shows an exemplary implementation of the suction detection algorithm using a threshold negative pressure at the pump inlet, and automated two-fold reduction in pump speed in response to this pressure limit. Red arrows indicate the instances of the algorithm detecting the pressure, and the immediate drop in flow and pump speed at this time.

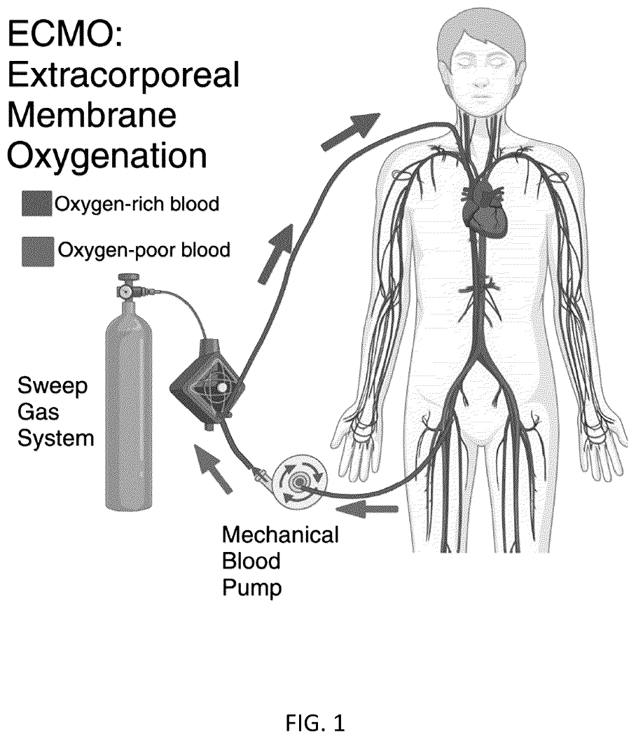

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending on the particular orientation of the figure. Similarly, if the device in one of the figures. is turned over, elements described as “below”, or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the invention.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Clinical blood pump consoles for extracorporeal membrane oxygenation (ECMO) are poorly accessible to researchers due to their high cost. Furthermore, clinical machines are built and designed at a high level of information security, which limits their integration with third-party machines and software. These barriers hinder researchers from customizing blood pump consoles for their investigational needs, limiting innovations and advancements in the areas of blood pump automation and pulsation.

In view of the foregoing shortcoming, one objective of this invention is to provide a programmable smart blood pump to address these needs. Another objective of the invention is to provide a method of operating the programmable smart blood pump for extracorporeal life support applications including, but not limited to, extracorporeal membrane oxygenation. The term “smart blood pump”, used herein the disclosure, refers to a blood pump system that is programmable for controlling pump speed and flow using existing clinical blood pumps.

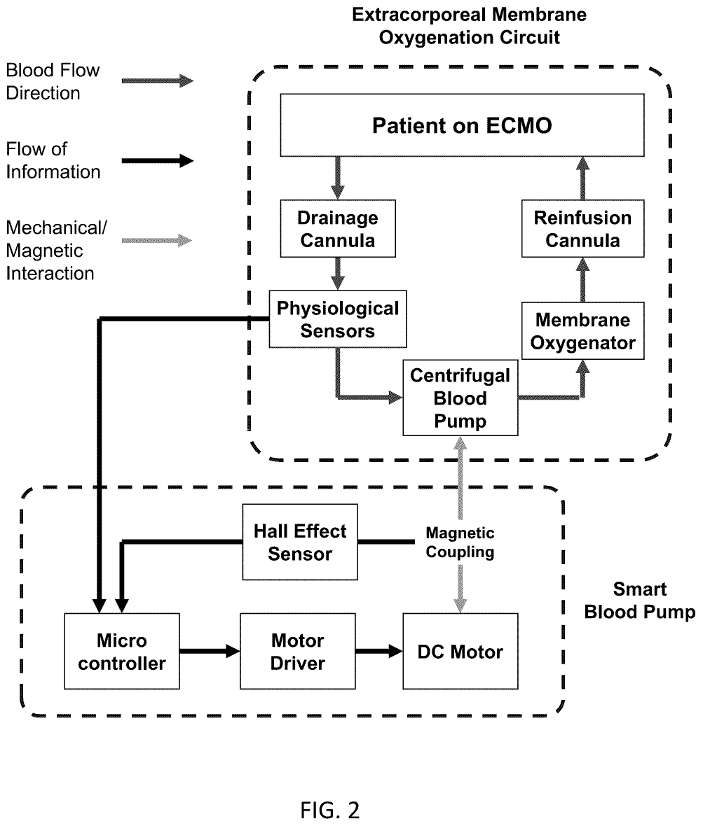

FIG. 2 shows schematically a smart blood pump according to embodiments of the invention. In this exemplary embodiment, the smart blood pump comprises a centrifugal blood pump coupled in an extracorporeal membrane oxygenation (ECMO) circuit and configured to provide a blood flow in the ECMO circuit for extracorporeal physiological support for a patient, a magnetic coupling disk coupled to the blood pump and configured to drive the blood pump to operably provide the blood flow, a motor coupled to the magnetic coupling disk and configured to drive the magnetic coupling disk, thereby driving the blood pimp, a motor driver configured to drive the motor, and a microcontroller configured to regulate a motor speed of the DC motor.

In some examples, the smart blood pump can provide constant or pulsatile blood flow, as well as receive physiological input to adjust pump rotational speed in a closed-loop feedback system. In some examples, the smart blood pump can receive user-scripted input to operate in a pulsatile/periodic manner. This pulsatility can be a clinically useful feature by better mimicking physiologic blood flow and pressure and may also improve blood washout. Moreover, the smart blood pump can serve as a low-cost research tool for pumping blood in a user-specified way. Another major design feature is physiologic feedback: the pump controller can receive a signal from another part of the extracorporeal circuit (such as pump inlet pressure) or a direct signal from the patient about their vital state (such as venous oximetry), which can be processed and analyzed to make proper adjustments to the pump speed.

According to the invention, the smart blood pump can be assembled for under $200 and uses open-source tools including Arduino. Using the smart blood pump console, centrifugal blood pump heads can be operated at clinically relevant levels of flow and pressure needed in extracorporeal life support applications (greater than 250 mmHg of the pressure head, greater than 4 L/min of the blood flow). Additionally, the programmable nature of the smart blood pump allows for utility beyond the standard indications of clinical extracorporeal blood pumps, including pulsatility and servo control. In some examples, the smart blood pump console can accommodate a wider range of clinical pump heads. According to the invention, the smart blood pump is an affordable, accessible platform to suit the varying needs of engineers and researchers for fostering innovations in ECMO technology.

In some examples, the pump control method prefers stability over rapid changes to best match the pump setting to the rate of oxygen consumption. To achieve this control, the pump motor speed automatically adjusts, using methods like proportional integral derivative scheme, to maintain a setpoint value for physiologic venous oxygen saturation. Gain scheduling can be in place so that the change in pump speed adjusts with the circuit resistance. An extracorporeal life support (ECLS) circuit that has a high resistance (e.g., small bore cannula) and/or at a high backpressure (such as veno-arterial circuit) elicits a smaller change in blood flow for a given change in the pump speed. Hence, these systems have higher gain coefficients than circuits with lower resistance that are more flow-sensitive to speed changes. Accordingly, the pump system incorporates pressure signals from the circuit into setting the ideal gain coefficients. Regardless of the mechanical profile of the circuit, the rate of change in pump speed cannot be too fast, which would otherwise elicit undesirable biological response due to blood trauma. The preliminary in vitro testing results suggest that the time window required for extracorporeal blood flow to fully mix with the native cardiovascular system can take between tens of seconds to several minutes. As such, a smart blood pump should not constantly adjust the speed in relations to oxygen level, as it can cause the pump speed to overshoot. Overshooting and oscillation in pump speed can lead to increased electrical current draw by the motor, excessive blood heating, and hemolysis. Thus, the invention only makes gradual adjustments in pump speed in response to variations in the patient oxygen status.

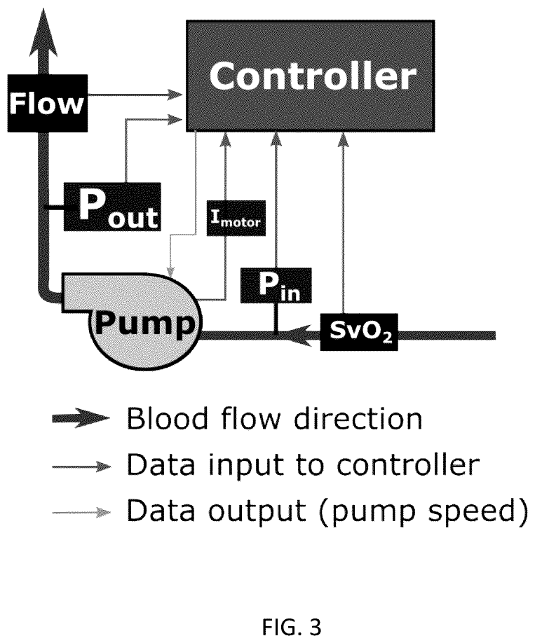

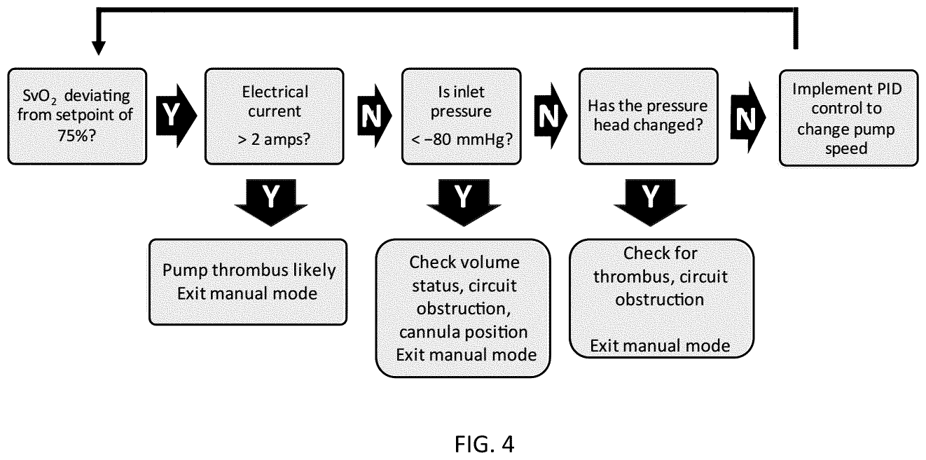

This feature of incorporating relevant circuit parameters also helps the system detect any problems inherent in the circuit that can interfere with optimal automation. Such parameters include circuit pressures, motor current, and oxygen saturation. These measures can be assessed by the described pump system to detect common mechanical issues like suction events when pressure gets too negative on the pump inlet, pump thrombosis when there is excessive current consumption, and/or circuit thrombosis when there is change in the circuit resistance profile. When these issues arise, the system exits from automatic mode and enters a manual mode until the issue is resolved. The technological approach of the invention is schematically shown in FIGS. 2-4, where FIG. 2 is a schematic diagram of a smart blood pump system, FIG. 3 is a schematic diagram of the smart blood pump incorporating circuit parameters to intelligently adjust the pump speed, and FIG. 4 is a flow chart for pump speed adjustment decision process in the smart blood pump system.

Initial prototyping and testing of the smart blood pump system have been done, including control over pulsatility of flow, the versatility of the smart blood pump to work across multiple types of magnetically driven centrifugal pumps, and proportional integral control scheme to user-specified input signal like flow. The smart blood pump can also be integrated with an oxygen sensor to enable automation according to the metabolic need of the patient.

System Assembly: FIG. 11 shows one exemplary embodiment (prototype) of the smart blood pump system-a programmable console for controlling pump speed and flow using existing clinical blood pumps. The prototyped pump system is built/assembled with a direct current (DC) motor, a magnetic coupling disk, a disposable pump head from Sorin/LivaNova, an Arduino Uno microcontroller, a motor driver shield, a potentiometer knob for regulating motor speed, and a Hall effect sensor for measuring rotational speed. The microcontroller in configured to regulate the motor speed. The magnetic coupling disk is used to rotate the pump impeller in a contactless manner. The external Hall effect sensor is positioned near the magnetic coupling to measure real-time rotational pump speeds. The smart blood pump is cross-compatible with LivaNova Revolution and Medtronic Affinity, which are standard ECMO blood pumps.

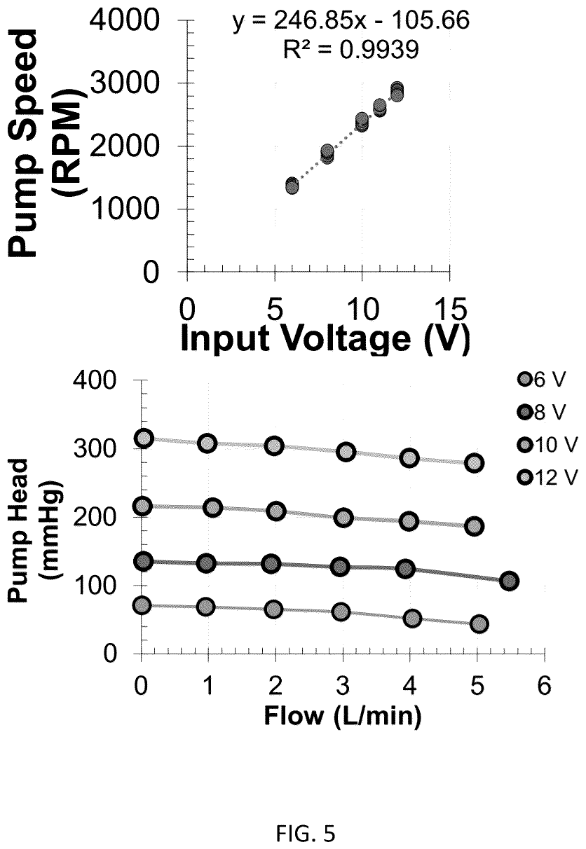

Pressure-Flow Curve: The ability of the prototyped blood pump system was evaluated to achieve clinically relevant pressure and flow by driving an ECMO blood pump. Sorin/LivaNova Revolution is one of the most used ECMO blood pumps on the market. Like most other clinical ECMO pumps, Sorin is a seal-less centrifugal pump that has magnetic impeller disc, such that the impeller can be driven in a contactless manner from an external pump driver. We created this motor driver using components listed above and quantified the levels of pressure and flow achieved with this assembly. For this purpose, a mock circulatory loop was built and tested with water and blood. Pressure differential between inlet and outlet, circuit flow, and pump speeds were recorded at varying outlet occlusion settings to yield a set of curves, commonly referred as an HQ curve. The pump could achieve pressure head of greater than 250 mmHg and flow greater than 4 L/min, which are more than sufficient metrics for most ECMO applications. These curves are shown in FIG. 5. This speed shows a linear response to an input voltage: for a voltage range of 6 to 12 volts, the LivaNova pump operates at 1300 to 3000 RPM (FIG. 5, top panel). The representative pressure-flow curves within this voltage range are shown in FIG. 5 (bottom panel). At these speeds, the pump easily achieves the level of flow and pressure head required in right-sided mechanical circulatory support. The blood pump also did not cause any significant damage beyond what was expected-normalized index of hemolysis was below 0.02 g/(100 L blood).

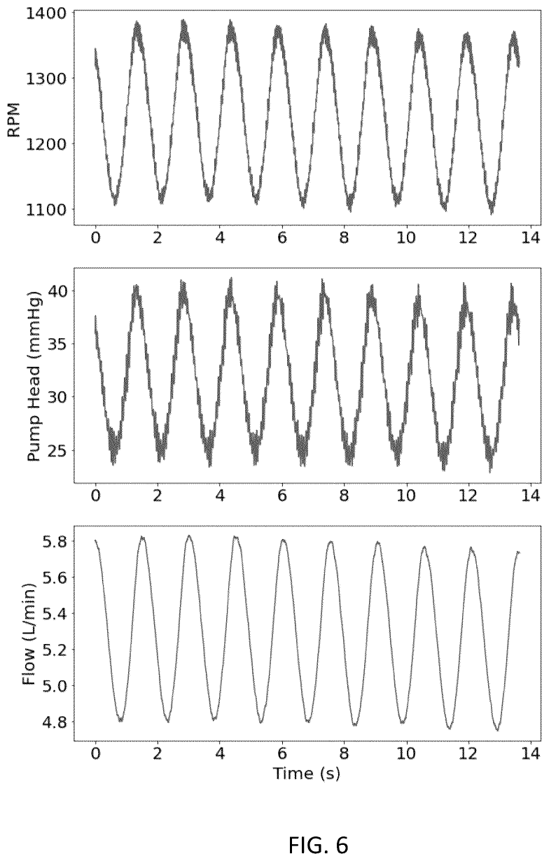

Pulsatility: The smart blood pump system can be programmed to vary its motor speed in a pulsatile manner. The sinusoidal variation was confirmed using the Hall effect sensor. Furthermore, it is confirmed that the modulated rotational speed in the motor was also reflected in the pulsatile nature of pressure and flow waveforms. FIG. 6 shows the representative plots demonstrating pulsatile RPM, pressure, and flow, i.e., the programmability of the pump with sinusoidal modulation of pump speed, pressure, and flow.

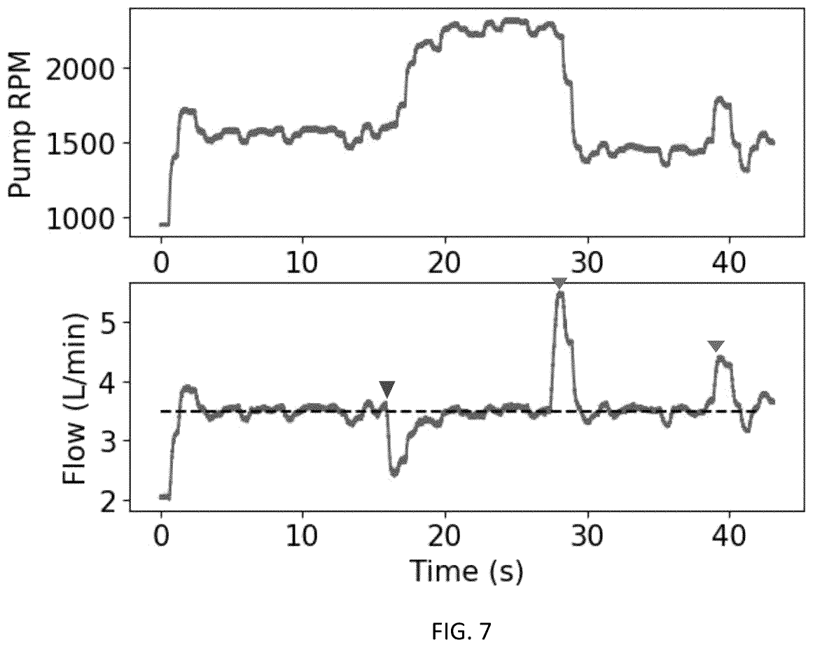

Pump automation: The smart blood pump system can be further programmed in Arduino or MATLAB® to enable proportional integral control for maintaining a setpoint flow. The setpoint flow maintenance was shown based on changes in the bias voltage and the circuit resistance profile. FIG. 7 shows a pump maintaining setpoint flow of 3.5 L/min in response to various flow disturbances. In some embodiments, the smart blood pump system can be integrated with an oxygen sensor to automate its function.

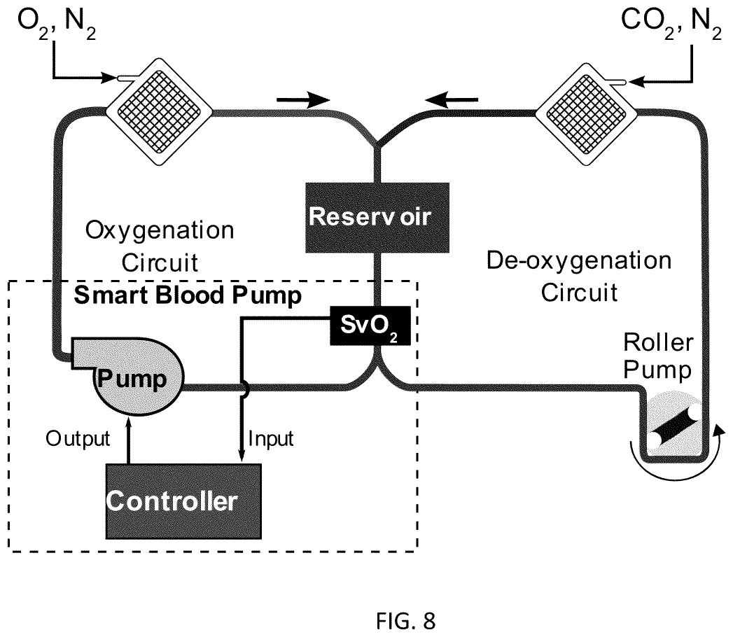

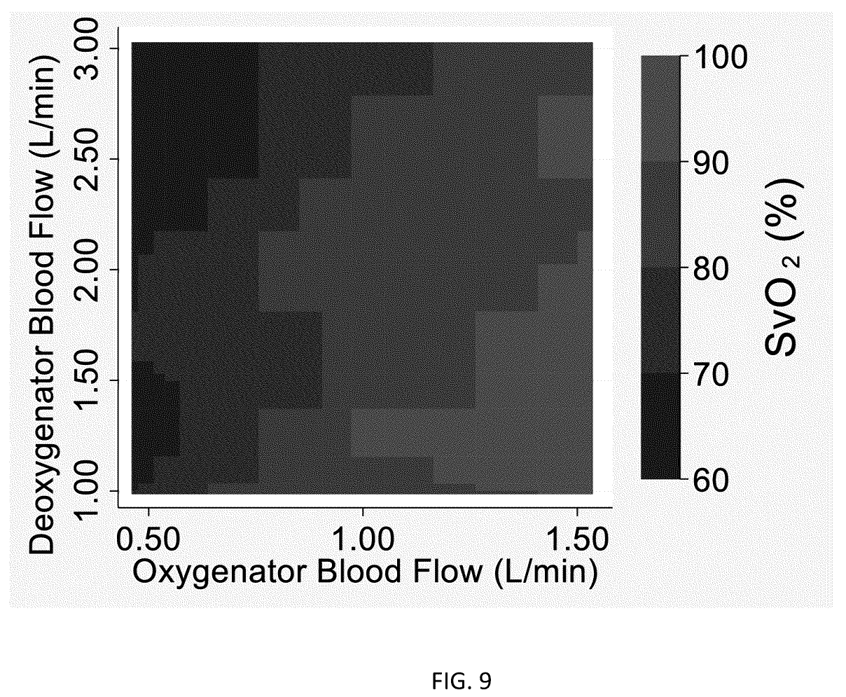

Oxygen Setpoint: A few benchtop experiments were conducted to characterize the open loop response between the blood pump and the circuit oxygen level. FIG. 8 shows the schematic for an ECMO mock loop circuit, FIG. 9 shows that varying levels of oxygen saturation can be attained, and FIG. 10 shows an oxygen saturation response curve to a step input voltage. The time constant for this change in oxygen saturation has been around 45-60 seconds, which leads the inventors to hypothesize that the pump speed is not rapid for maintaining homeostatic oxygen balance.

Blood Oximetry: A robust dataset was collected from a large animal disease model of right heart failure. This model has shown that mixed venous oxyhemoglobin saturation correlates with right heart function. Clinical observational data from pulmonary hypertension patients, one of the intended disease applications for our invention, also show that mixed venous oxygen saturation (SvO₂) is a strong predictor of right heart failure and other adverse clinical outcomes. The SvO₂ is a measurement of the amount of oxygen in the blood that returns to the heart after the body's tissues and organs have consumed oxygen. It is a key indicator of how well oxygen is being delivered and used throughout the body. Other chronic lung diseases also show this similar trend, suggesting that venous oxygen can serve as a biomarker basis for titrating the smart blood pump.

In some examples, the smart blood pump system also includes a feedback mechanism for pump speed control by using oxygen saturation as a surrogate measure for patient status and basis for speed adjustment. Specifically, the smart blood pump is integrated with an oxyhemoglobin monitor. The experimental platform described in FIG. 8 is used to evaluate the closed loop response. Once this feedback loop is closed, the system is refined by iteratively testing on the bench and later in animals in an acute setting, which further drills down on the optimal rate of pump speed adjustment to achieve oxygen maintenance without the excessive current draw, heating, and/or blood hemolysis.

In some examples, a safety mechanism is also incorporated into the smart blood pump system to ensure that the pump does not blindly increase speed in response to mechanical complications of the circuit, such as circuit obstruction, low fluid volume status, and clot formation. This will entail incorporating other metrics of the circuit performance, such as circuit pressures and electrical current consumption, to detect abnormal ECMO circuit condition. A flow chart in FIG. 4 shows a step-by-step process including these troubleshooting methods. There are safety checks to screen for problems like pump thrombosis that can be detected from elevated motor current; suction events and cavitation secondary to cannula malposition and low volume status that can be detected from pump inlet pressure; and oxygenator thrombosis that can be detected from increased blood flow resistance.

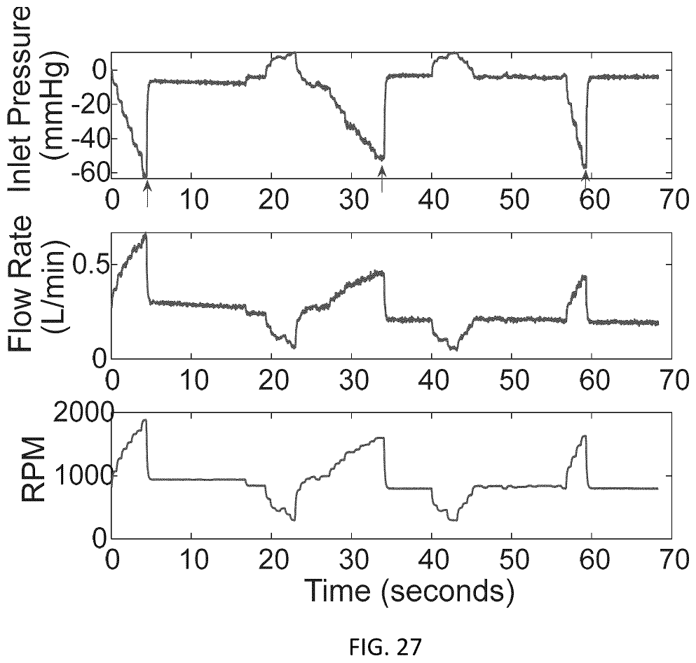

FIG. 27 illustrates an example of the suction detection algorithm implemented by setting a lower-limit value for pump's inlet pressure. More negative this inlet pressure gets, the closer the pump gets to a suction event. In this implementation, the pump voltage is automatically reduced to half when the inlet pressure gets below a certain negative value. These events are marked as red arrows in the accompanying figure, at which point the accompanying RPM gets reduced by a factor of 2. Accordingly, we can use pressure values at varying points along the circuit to diagnose different types of circuit complications. Pump thrombosis can be diagnosed by abnormally high level of current consumed by the motor, as higher torque is required to rotate the impeller with a clot burden's presence. Oxygenator thrombosis can be diagnosed by looking at the pressure difference between the inlet and the outlet and dividing the difference by the flow rate to calculate oxygenator resistance. Any abrupt increase in resistance is an indicator for thrombus burden within the device.

As a part of the integration effort, the smart blood pump also includes a safety mechanism to prevent any occurrences of unwanted speed increase that can lead to adverse events such as cavitation. These safety limits also help prevent error accumulation that may occur in the event of circuit occlusion that may prevent the system from achieving a setpoint oxygen saturation level. FIG. 4 shows a flowchart that checks for venous oxygen saturation while also handling mechanical anomalies such as cavitation and circuit obstructions.

To verify this safety setup, the pump is placed in a compact circuit flow loop, and its speed is initially set at 1500 RPM but varied in flow between 1, 2, and 3 L/min. The pump inlet is progressively occluded until cavitation pressure is reached to confirm whether the system automatically detects suction. Using the same circuit setup, the pump outlet is also occluded to verify whether the pump detects high circuit resistance, which is calculated by the pump pressure head divided by flow. This pump pressure head can vary by the mode of circuit support (veno-venous and veno-arterial circuits have different afterload requirements) as well as disease indication (e.g., COVID patients require a high blood flow support compared to COPD patients, which require higher level of pressure generated). The safety limit can therefore be set a prior based on expected level of flow support needed and adjusted thereafter.

Oxygen Setpoint Maintenance, In Vitro: Upon integration between the pump and the oxygen sensor, the combined functionality was verified in vitro using the established dual blood circuit setup. The schematic diagram of the dual blood circuit is shown in FIG. 8. The circuit on the left side includes the smart blood pump-driven, oxygenation circuit that has a gas exchanger with a blend of oxygen and air as its sweep gas. The deoxygenation circuit on the right side is driven by a positive-displacement roller pump and gas exchangers using a mixture of 92% N₂ and 8% CO₂ as sweep. The gas exchangers in oxygenation and deoxygenation circuits both are standard membrane oxygenator used in clinical settings, such as Maquet Quadrox or MC3 Nautilus. The sweep gas flow rate for both gas exchanger units is maintained between 0.5 and 1 times the blood flow rate for a target mixed CO₂ partial pressure of 30 to 50 mmHg. The real-time mixed venous hemoglobin (SvO₂) reading is sampled right before the circuit splits, to ensure sufficient mixing between deoxygenated and oxygenated blood.

For control algorithm employing SvO₂ maintenance, the SvO₂ setpoint is set at 75% as a default physiological target value. The circuit blood volume starts at 1000 mL. The smart blood pump employs PID control and tuned for its gain coefficients. Then, the smart blood pump is evaluated against two different oxygen disturbance scenarios: 1) varying the oxygen concentration of the sweep gas, and 2) circuit flow disturbance. In scenario #1, the oxygenator sweep gas is mixed with nitrogen to achieve oxygen % between 10% and 100%. In scenario #2, the oxygen % is fixed, but the circuit blood flow is varied using an adjustable clamp while operating within the pressure limit set by the safety mechanism.

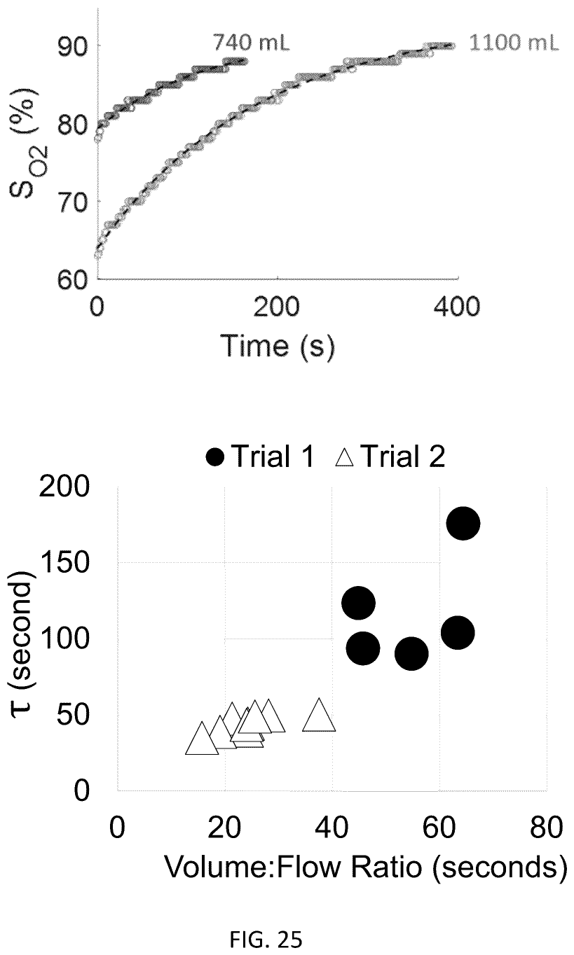

Preliminary investigations characterized the open-loop response of the in vitro ECMO flow circuit between the input signal to the pump and the resulting oxygenation trajectory (FIG. 25). For a given step change in the motor voltage and speed, the corresponding real-time change in oxygenation can be modeled as an exponential function of time t with the form: S_{O2}(t)=a₁−a₂ exp (−t/τ), where τ is the time constant that represents the amount of time required to elicit 63% of the total change. For this benchtop platform, t had a direct relationship with the circuit blood volume, meaning that a bigger volume system needs longer time to equilibrate. Further, we observed that the ratio between the volume and the pump's blood flow rate had a direct relationship with t. The circuit blood volume can be thought of as a parameter representing patient size and blood volume contained within their body. The volume-to-flow ratio can be thought of as the time it takes for blood to make a full circulation throughout the circuit. In other words, the ideal control strategy will account for the blood volume, the current blood flow rate, and accordingly the time it takes for the blood to fully mix and the oxygenation to settle in order to minimize unstable pump controller behaviors such as oscillations and overshoots. Other patient parameters of importance include underlying disease etiology and severity, the level of metabolic/exercise activity (e.g. sedentary vs ambulatory use of ECMO), and the surgical attachment mode (i.e. veno-venous versus veno-arterial modes).

In a similar vein, the smart blood pump control algorithm needs to account for device specifications and operation parameters. Since each pump has its unique rotational performance and pressure-flow curves, the same input voltage will yield varying operational outputs. Additionally, oxygenators and cannulas have their blood flow resistance profiles. Smaller bore cannulas will add more resistance to the circuit than the larger-bore cannulas, meaning that the pump will need to add more pressure in order to achieve the same level of flow rate as the smaller bore cannulas. Accordingly, these device selections will need to inform the control strategy in order to ensure stable and rapid performance.

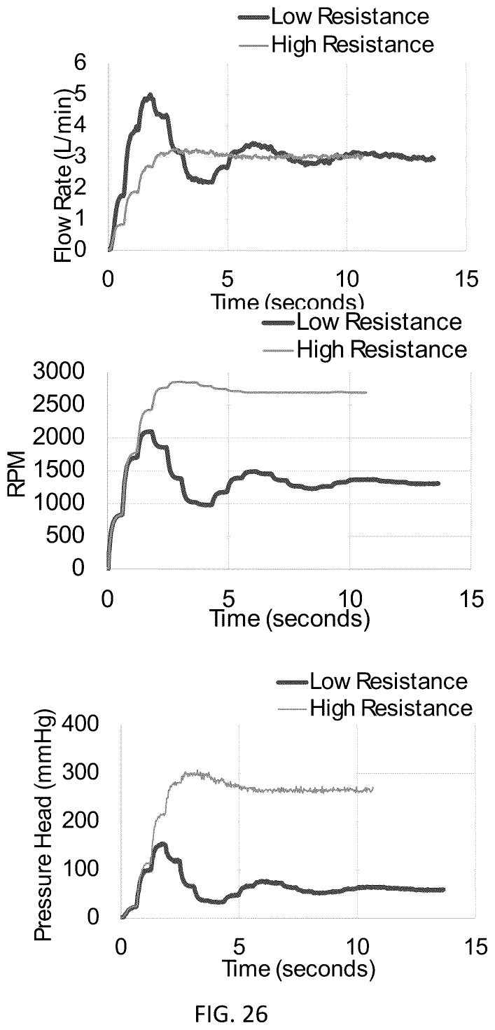

To illustrate this point, FIG. 26 provides data showing how circuit resistance profile affects the stability of automated controller. Two scenarios are shown: low resistance and high resistance. The same sets of target flow rate and PI gain coefficients are used for both, but the performances are markedly different. In the low-resistance scenario, we see an overshoot and some oscillations before settling at the steady-state values of pump speed of 1300 RPM, pump head of 60 mmHg, and flow of 3 L/min. For the high resistance scenario, there is a slower yet steadier increase in the pump RPM with little to no overshoot before arriving at 2700 RPM pump speed, and accompanying pump head of 265 mmHg and the same setpoint flow of 3 L/min. Higher gain values lead to overshooting behaviors in a low-resistance circuit as shown here, as there is a greater change in flow for a given change in the motor input voltage. In clinical situations, the proper selection of gain coefficients needs to be informed by the total resistance of the circuit to ensure stability in automated control. The total resistance of the circuit will be determined by the selected cannulas' bore size and length, and the oxygenator model. Likewise, the attachment mode of ECMO will also affect the backpressure of the circuit. A circuit that reinfuses blood into the arterial system will have a higher afterload than a circuit that returns blood to the venous side, which will accordingly increase the pressure requirement.

Oxygen Setpoint Maintenance, In Vivo: The smart blood pump is also evaluated in vivo. A sheep is anesthetized and surgically prepped for cannulation. Depending on the disease model, we will change the cannulation strategy. In one embodiment, we focus on the application for pulmonary hypertension/right heart failure model, so we can employ a central veno-arterial circuit or right atrium-to-left atrium configuration. The circuit uses a Maquet Quadrox or an MC3 Nautilus as the oxygenator. Pure oxygen is used as the oxygenator sweep gas and maintained at 1 L/min, and CO₂ is adjusted using the oxygenator sweep gas flow rate. The ECMO circuit blood flow is manually started at 1 L/min. The activated clotting time is kept between 180 and 220s using intravenous heparin infusion to model standard clinical ECMO anticoagulation. The SvO₂ sensor is placed on an ECMO tubing before the smart blood pump.

The circuit blood flow starts at 1.0 L/min. A vascular occluder cuff is also placed around the pulmonary artery to model acute right heart failure. The cuff can be controlled for tightness by injecting or drawing back its fluid content. Then for the following 6 hours, there are periodic disturbances in the pulmonary artery, and in mixed venous saturation, using this cuff. Every 30 minutes, the cuff either is injected with a fixed volume amount or emptied completely. The test also alternates between 1 hour of manual correction by a blinded ECMO specialist and 1 hour of automatic control to allow for a back-to-back paired comparison between control schemes. In either automatic or manual approach, the goal is to be within 5 percentage points from the setpoint SvO₂ of 75%. A 2-hour segment of the timeline has 2 full cycles of cuff occlusion/dilation and 1 full cycle of manual/auto.

Without intent to limit the scope of the invention, exemplary embodiments of the invention are described below.

In one embodiment shown in FIG. 2, the blood pump system includes a blood pump configured to provide a blood flow in an extracorporeal membrane oxygenation (ECMO) circuit for extracorporeal physiological support for a patient; a magnetic coupling member configured to drive the blood pump to operably provide the blood flow; a DC motor configured to drive the magnetic coupling member, thereby driving the blood pump; and a controller that can have a stored program script to regulate a motor driver that transmits electrical input (current and voltage) to the DC motor.

In one embodiment, the blood pump is a standard ECMO blood pump such as Sorin/LivaNova, or a customized blood pump.

In one embodiment, the blood flow is a constant blood flow, or a pulsatile blood flow. According to the invention, the pulsatility can be adjusted.

In one embodiment, the magnetic coupling member is configured to rotate a pump impeller of the blood pump in a contactless manner.

In one embodiment, the blood pump system further comprises a Hall effect sensor positioned in relation to the magnetic coupling member to measure a pump speed of the blood pump in real time. The controller is further configured to receive a signal of the pump speed from the Hall effect sensor and regulate the motor speed of the DC motor responsively.

In one embodiment, the blood pump system further comprises a physiological sensor such as an oxyhemoglobin sensor disposed in the ECMO circuit for monitor physiological parameters of the patient.

In one embodiment, the controller is further configured to receive a physiological input from the physiological sensor in the ECMO circuit and regulate the motor speed responsively, thereby adjusting the pump speed in a closed-loop feedback system.

In one embodiment, the controller is further configured to receive a signal from the patient about its vital state and regulate the motor speed responsively, thereby adjusting the pump speed.

In one embodiment, the controller is further configured to receive a user-scripted input to operate the blood pump system in a pulsatile or periodic manner.

In one embodiment, the blood pump system is programed to vary the motor speed in a constant manner or a pulsatile manner.

In one embodiment, the blood pump system is programed to follow a setpoint value to correct homeostasis.

In one embodiment, the blood pump system further comprises a feedback mechanism for controlling the pump speed by using venous saturation as a surrogate measure for patient status and basis for speed adjustment, so as to enable automation according to metabolic need of the patient.

In one embodiment, the feedback mechanism comprises a venous saturation monitor including an oxygen sensor coupled in the ECMO circuit to monitor an oxygen level, and an oxygen consumption, and/or mixed venous oxygen saturation (SvO₂) of the patient.

In one embodiment, the blood pump system is programmed, such that the pump speed is automatically adjusted using a proportional integral derivative scheme to maintain a setpoint flow for physiologic venous oxygen saturation.

In one embodiment, the blood pump system further comprises a safety mechanism for ensuring the blood pump not to blindly increase speed in response to an abnormal ECMO circuit condition including circuit obstruction, low fluid volume status, clot formation, a suction event, pump thrombosis, and/or circuit thrombosis.

In one embodiment, the blood pump system is operable at clinically relevant levels of flow and pressure needed in extracorporeal life support applications, for example, with greater than 250 mmHg of the pressure head, and greater than 4 L/min of the blood flow.

In one embodiment, the method of operating the blood pump system for extracorporeal life support applications comprises driving the DC motor, thereby the blood pump at a target pump speed to provide the blood flow in the ECMO circuit for extracorporeal physiological support for the patient in an automatic mode or a manual mode, wherein the target pump speed is determined based on preset operation parameters.

In one embodiment, the preset operation parameters include setpoint flow and/or pressure in the ECMO circuit, motor current, oxygen saturation, and/or a physiological state of the patient.

In one embodiment, the physiological state of the patient includes an oxygen level, cardiac output, an oxygen consumption, and/or mixed venous oxygen saturation (SvO₂).

In one embodiment, other preset, patient-specific operation parameters include patient body weight, body surface area, estimated blood volume, disease etiology and reason for extracorporeal physiological support, the attachment mode of the circuit (e.g. veno-venous or veno-arterial), and whether the patient is using for sedentary or ambulatory application.

In one embodiment, the preset, device-specific operation parameters include the models of pump, oxygenator, and cannulas; pump pressure-flow curves; bore size of the cannula and the cannula resistance; and oxygenator resistance.

In one embodiment, the method further comprises measuring the pump speed of in real time; and regulating the motor speed responsively to adjust the pump speed when the measured pump speed is deviated from the target pump speed.

In one embodiment, the method further comprises measuring the SvO₂; and regulating the motor speed responsively to adjust the pump speed when the measured SvO₂ is deviated from a setpoint value.

In one embodiment, the method further comprises measuring the flow rate and/or pressure in the ECMO circuit; and regulating the motor speed responsively to adjust the pump speed when the measured flow rate and/or pressure are deviated from the setpoint flow and/or pressure.

In one embodiment, the method further comprises detecting an abnormal ECMO circuit condition including circuit obstruction, low fluid volume status, clot formation, a suction event, pump thrombosis, and/or circuit thrombosis; and regulating the motor speed responsively to ensure the blood pump not to blindly increase speed when the abnormal ECMO circuit condition is detected.

In one embodiment, when the abnormal ECMO circuit condition is detected, the blood pump system exits from the automatic mode and enters the manual mode until the abnormal ECMO circuit condition is resolved.

In one embodiment, the invention relates to automate pump operation based on important circuit/patient parameters to better address their physiologic deficits, that matches with the patient's rate of oxygen consumption.

In one embodiment, the invention also relates to a method that prioritizes making moderate pump speed changes to avoid rapid changes and flow oscillation.

According to the invention, the smart blood pump can accommodate various types of clinical blood pump heads, whereas most clinical pump consoles do not accommodate other companies' products.

According to the invention, the smart blood pump system is a low-cost research tool that can be readily programmed and implemented for each researcher's needs.

Among other things, the invention may find the following practical applications.

Feedback Control: A major problem in the current ECMO landscape is the lack of automated feedback control especially for blood pumps. The pump speed dictates the amount of cardiopulmonary support provided to the patient. There are many instances in which the blood pump setting needs to be adjusted. For example, when the pump speed is too high compared to the patient's fluid volume status or the malpositioning of the cannula that limits blood drainage, suction can occur, which can lead to cavitation, flow restriction, and hemolysis. The pump speed has to be reduced in response to better match the speed to volume/drainage status, which can change drastically in critical care situations. Another common issue is the mismatch between ECMO pump speed and the patient's metabolic activity. When a patient ambulates during ECMO for physical rehabilitation and strength training purpose, patient's heart rate, stroke volume, and/or cardiac output increases in response to the body's elevated rate of oxygen consumption needed to keep up with physical exercise. In the current ECMO landscape, the blood pump speed has to be manually increased in response to this elevated physical activity. Without constant monitoring, these pumps may not properly accommodate patient's specific metabolic and physiologic needs. Allowing the pump to self-adjust to these cues will be beneficial on multiple fronts. First, it will facilitate the use of pump in mobile applications such as patient transport, physical rehabilitation, and exercise. Second, it will help maintain an optimal pump speed in response to other sources of disturbances to the circuit operation to minimize adverse occurrences of suction and hemolysis. Finally, it also means that ECMO as a therapy will become less labor-intensive due to the reduced monitoring requirement.

On a broader scale, smart blood pump algorithm can be used to automate applications outside of ECMO, most notably ventricular assist devices (VADs). Despite their longer history and successes in long-term use compared to ECMO, VADs also lack feedback control mechanism. There are numerous research papers noting that VADs are not well-designed to address exercise intolerance in patients because they do not have automatic speed adjustments. Our invention can apply to VAD settings to offset physiologic deficits that arise during exercise. We also envision this similar technology can be applied to automate dialysis toward a wearable application.

Pulsatility: While the benefits of pulsatile versus constant flow remain to be settled, many researchers and clinicians have argued for its benefit as it better matches cardiovascular physiology. Some of the implantable ventricular assist devices also have the pulsatility function built in, namely HeartMate III. Another potential benefit of pulsatility is a better wash-out profile, meaning that pulsed flow may help clear out stagnant areas that may otherwise accumulate cells and clotting factors that contribute to clot propagation. The strengths of our invention are the greater degree of control over the pulsatile settings, and the ability to integrate with existing pump technologies like what we show here.

Cost: The invention also offers a robust, low-cost research platform for researchers who may not have easy access to clinical pump consoles. The presented prototype here was constructed under $200.

Without intent to limit the scope of the invention, examples and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE

### Smart Centrifugal Blood Pump Platform for Extracorporeal Membrane Oxygenation

In this exemplary study, we designed and built a low-cost Smart Blood Pump platform that can operate a clinical centrifugal pump at hemodynamic conditions relevant to extracorporeal applications. The programmable feature is also demonstrated by pulsatility and a simple servo control for maintaining flow. As the first pass, this work focused on one specific type of pump head-LivaNova Revolution. This prototype effort serves as the first iteration toward ultimately establishing a universal pump console that is affordable, operable across various types of pump heads, and programmable to fit the needs of engineers and clinicians.

### Hardware

Many of the clinical centrifugal blood pumps used for ECMO, including Medtronic Affinity and LivaNova Revolution, have impeller blades that are embedded with magnets to enable their contactless magnetic drive. The prototype for our Smart Blood Pump console includes a direct current (DC) permanent magnet motor, a magnetic disc that drives the pump impeller, and a custom-designed, laser-cut, wooden enclosure that houses the motor and the magnetic disc, as shown in FIG. 11. The enclosure is designed to ensure proper alignment and coupling between the magnetic disc and the pump impeller. The pump can be controlled manually by connecting the motor to a direct current variable power supply. For advanced, programmable flow control, the motor can be connected to an Arduino-based microcontroller and a motor driver for regulating the motor speed in a programmed way. For these intentions, we provide Arduino scripts that demonstrate pulsatility and servo flow control (Table 1).

### Design Files

TABLE 1
Design files for laser cutting and
programming pump operation via Arduino

| | | Open | |
|---|---|---|---|
| | File | source | |
| Design file name | type | license | Location of the file |
| laser_cut_ | .dwg | CC BY | Zenodo: |
| enclosure.dwg | | 4.0 | https://doi.org/10.5281/ |
| | | | zenodo.12555206 |
| sinusoid_pulsatile_ | .ino | CC BY | Zenodo: |
| flow.ino | software | 4.0 | https://doi.org/10.5281/ |
| | (Arduino | | zenodo.12555206 |
| | IDE) | | |
| PID_flow_ | .ino | CC BY | Zenodo: |
| control.ino | software | 4.0 | https://doi.org/10.5281/ |
| | (Arduino | | zenodo.12555206 |
| | IDE) | | |

### Materials

TABLE 2
Bill of Materials Summary for Enclosure Assembly

| | | | | Total | | |
|---|---|---|---|---|---|---|
| | | | | Cost- | Source of | Material |
| Designator | Component | Quantity | Cost per unit | Currency | Materials | Type |
| Enclosure | Plywood | 1 | $4.60/count | $4.60 | Provided by | Organic |
| | Sheets For | | | | Vanderbilt | Polymer |
| | Laser Cutter | | | | Wondr'y | |
| | 12″ × 24″ × | | | | Center. | |
| | ⅛″ | | | | Materials | |
| | | | | | also on | |
| | | | | | Amazon | |
| Enclosure | DC Motor, | 1 | $18.69/each | $18.69 | Amazon | NA |
| | XD-3420 | | | | | |
| Enclosure | Magnetic | 1 | $101.67/each | $101.67 | McMaster | Metal |
| | Coupling | | | | Carr | |
| | Shaft | | | | | |
| | (9199T2), | | | | | |
| | 1-3/8″ Overall | | | | | |
| | Length, | | | | | |
| | 1-31/32″ OD | | | | | |
| Electrical | SHIELD- | 1 | $14.50 | $14.50 | Maker | NA |
| | MD10 Cytron | | | | Motor | |
| | 10A Motor | | | | | |
| | Driver Shield | | | | | |
| Electrical | SparkFun | 1 | $21.50 | $21.50 | SparkFun | NA |
| | RedBoard— | | | | | |
| | Programmed | | | | | |
| | with Arduino | | | | | |
| Total | | | $160.96 | | | |

### System Assembly

This enclosure includes seven laser-cut panels-pump interface, motor shaft, motor base, motor cords, side panel (×2), and base panel. The panel dimensions were selected to provide the most optimal compatibility with the LivaNova Revolution pump. To allow the components to interlock, dents and holes were cut into the panels, as shown in FIGS. 12-13. The base panel is used for stabilizing the motor and have openings to insert the side panels. The distance between the magnet and the pump head should be maintained at 0.5 cm, so the total height of the side panels should account for the combined lengths of the motor, the shaft, and this ideal interface spacing. The pump interface panel's cut-out should be large enough to accommodate the pump diameter.

For assembly, first insert the two side panels onto the base layer panel. Between the two side panels, stack the motor base panel on top of the motor cords panel. The DC motor body was then secured within motor base and motor cords panels. The motor shaft was fitted through the hole on the center motor shaft panel and was directly coupled to the magnetic disc using provided set screws. The top interface panel was then fitted onto the top indentations of the two side panels. The pump head was placed on the top panel and secured with three sets of bolts. FIGS. 14-15 show the completed assembly with the panels labeled. The enclosure's pump interface panel, where the pump head is secured, was designed to be modular and generally compatible with magnetically driven centrifugal pump heads. Later we will discuss additional changes that need to be made to accommodate other pump types.

### Electronic Setup

A Hall effect sensor can be used to monitor the real-time rotational speed of the magnetic disc. In one embodiment, the sensor is fixed within an activating distance of 13 mm to the magnetic disc. The red wire from the Hall effect sensor is connected to a supply voltage of 5 volts, the black wire is connected to ground, and the blue wire is connected to a data acquisition system, (FIG. 16, see later for connecting to the PowerLab system). This blue data output wire is also connected to a pull-up resistor of 200 ohms to the same 5-volt voltage supply.

The motor can be directly connected to a DC variable power supply to manually operate the pump. The positive (red) and negative (black) terminals from the supply are connected to the positive (red) and negative (black) leads of the motor, respectively, to rotate the magnetic disc in the direction of the LivaNova Revolution's blood flow, as shown in FIG. 17.

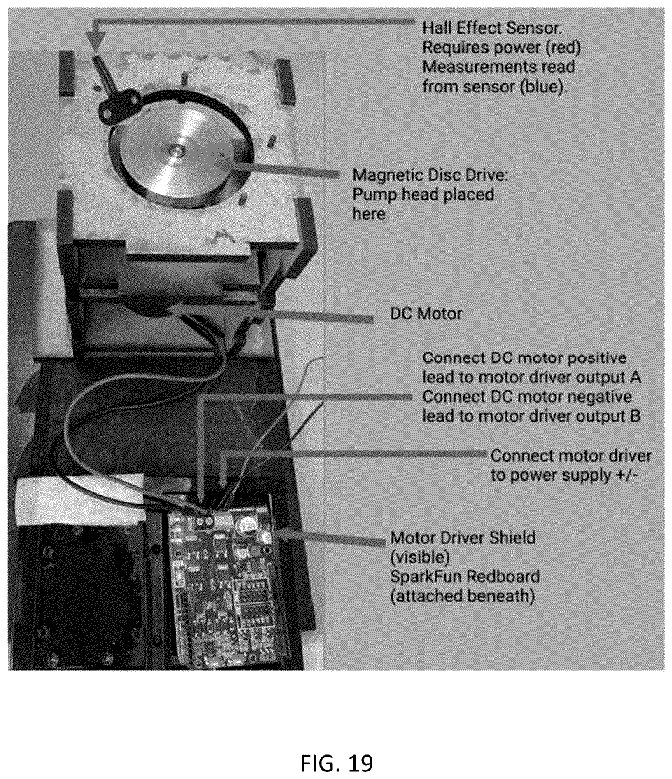

FIG. 18 shows how the pump can be connected to the Sparkfun Redboard microcontroller for programmable flow. A Cytron motor driver shield was attached on top of the Redboard. To match with the blood flow direction of the LivaNova Revolution, the DC motor's positive red terminal lead is attached to the “outA” screw terminal pin of the motor driver shield, and the DC motor's negative black terminal lead is attached to the “outB” screw terminal pin. Also on the motor driver shield, locate the green screw terminals of the shield labeled “POWER” and its positive and negative terminals. Connect the positive screw terminal from the shield to the positive terminal of the power supply, and the negative screw terminal from the shield to the negative terminal of the power supply. FIG. 19 shows the bird's eye view of the programmable flow setup. Then, proceed to compiling and uploading an Arduino script to the Redboard for programmable operation of the pump.

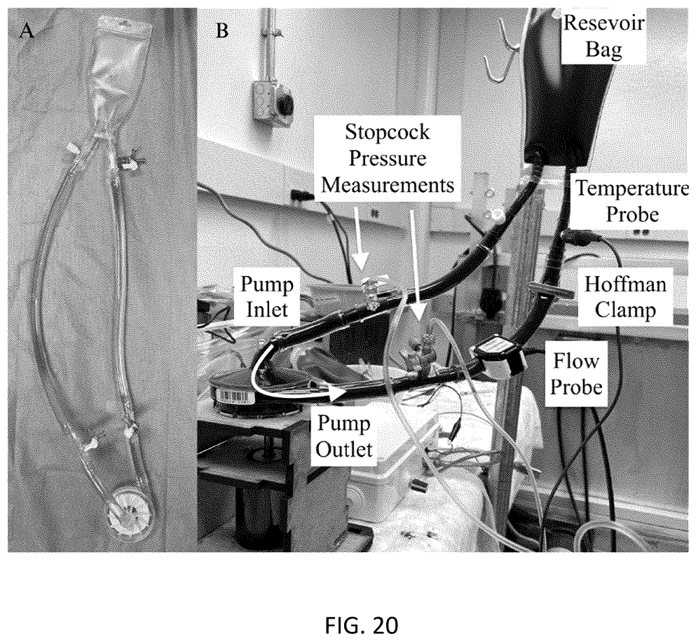

Flow Circuit Loop: The basic pump functionality can be confirmed using a simple flow loop circuit. The circuit design was published previously and includes the following components in the specified order of assembly (FIG. 20, panel A, Table 3): a two-port 100 mL reservoir bag, ¼″ to ⅜″ adapter with a luer sideport, 6″ of ⅜″ tubing, ⅜-⅜ connector with a luer sideport with a stopcock for pump inlet pressure, 6″ of ⅜″ tubing, LivaNova pump head, 6″ of ⅜″ tubing, ⅜-⅜ luer connector with a luer sideport and a stopcock for pump outlet pressure, 6″ of ⅜″ tubing, ¼″ to ⅜″ adapter with a luer sideport and a temperature probe (FIG. 20, panel B). Prime the circuit with at least 200 mL of fluid, which could be saline, 40% glycerol solution to match the viscosity of blood, or whole blood if there is proper biohazard handling plan in place at your research site. Use the two three-way stopcocks within the flow circuit loop as access points for syringes to push the fluid in and to evacuate air out of the circuit. Once the circuit is primed and free of air bubbles, position the reservoir bag at an elevated height. An adjustable Hoffman clamp should be placed distally to the pump outlet pressure port for controlling the pump afterload. A Transonic flow probe can also be positioned, matching the direction of flow, to acquire a real-time flow signal.

TABLE 3
Summary of items for constructing the flow loop circuit.

| | | | Source of | Material |
|---|---|---|---|---|
| Designator | Component | Quantity | Materials | Type |
| Circuit | Glycerol* >99% | 1000 mL* | Fisher | glycerol |
| Material | | | Chemical | |
| Circuit | Normal saline* | 1000 mL* | ICU | physiologic |
| Material | | | Medical | saline |
| Circuit | Two Port 100 mL | 1 per circuit | Qosina | polymer |
| Material | Reservoir bag | | | |
| Circuit | ¼″ to ⅜″ luer | 2 per circuit | Qosina | polymer |
| Material | adapter | | | |
| Circuit | ⅜″ to ⅜″ luer | 2 per circuit | Qosina | polymer |
| Material | adapter | | | |
| Circuit | 6″ of Tygon tubing | 4 segments | VWR, | polymer |
| Material | (inner diameter = | | catalog #: | |
| | 3/8″, wall | | 89404-282 | |
| | thickness = 3/32″) | | | |
| Circuit | Three-way | 2 per circuit | Qosina | polymer |
| Material | Stopcock | | | |
| Circuit | LivaNova | 1 per circuit | eSutures | polymer |
| Material | Revolution Pump | | | |
| | Head | | | |
| Circuit | Hoffman clamp | 1 per circuit | McMaster- | Metal |
| Material | | | Carr | |

Asterisks (*) indicate that the exact volume will need to be adjusted according to the circuit volume as well as the target viscosity of the fluid.

### Operations

Manual Flow Mode: After the circuit is primed and the motor is connected directly to a DC power supply as shown in FIG. 17, set the output voltage to a low level of 3 to 4 volts. Ensure that the motor and the pump are rotating in alignment with the flow direction of the LivaNova Revolution pump. Then, adjust the power supply voltage according to the target flow, pressure, and/or rotational speed. Increase voltage in increments of 0.1 to 1 volt to ensure a smooth change in speed. A large step change can yield a spike in the motor current that can lead to magnet decoupling.

Programmable Flow Mode: Navigate to Arduino IDE on a computer. IDE is available as an app or web-based interface (https://www.arduino.cc/en/software). Connect the RedBoard to the computer using the provided USB cable, and then select your board and port connection using the options from the IDE tools menu. We provide two different Arduino scripts to demonstrate programmed flow-pulsatile flow and servo control for flow adjustments (Table 1).

For pulsatile operation, compile and upload the Arduino script named “sinusoid_pulsatile_flow.ino” (Table 1). The script uses Arduino's digital output pulse width modulation (PWM) to vary the input voltage to the motor. The PWM output value ranges from 0 to 255, where 0 represents zero voltage and 255 represents the full voltage equal to the power supply voltage. Pulsatile control can be achieved by changing the minPWM and maxPWM variables defined in the Arduino script. The difference between the two variables sets the pulse height. Additionally, the time duration of a full pulsatile cycle needs to be defined under the variable name period in the unit of milliseconds. For a default setting, we set period of 1000 millisecond, minPWM of 150, and maxPWM of 200. We caution the readers to not make the period too short, and to not make the pulse height too high when they are just starting out. Either of these changes will require rapid change in motor speed that can lead to magnetic decoupling. It should be appreciated that these settings can be incrementally changed from the provided default values.

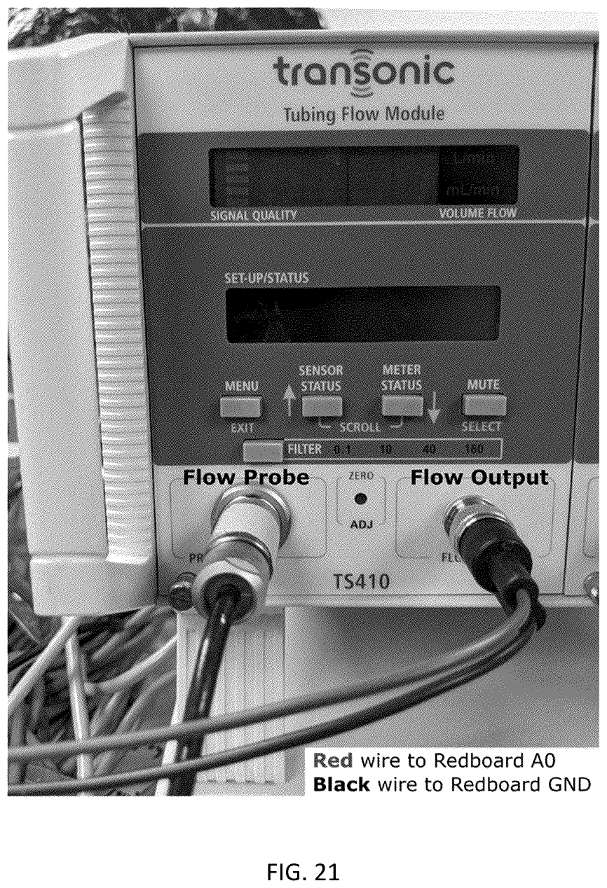

Servo Flow Control: To close the feedback loop between the Redboard and the flow sensor, a Transonic flow probe is attached onto one of the ⅜″ segments of the flow loop circuit. Ensure that the flow direction indicated on the probe matches with that of the circuit. Connect the other cable connection end of the flow probe to a pin labeled “PROBE” on the front panel of a Transonic flow meter (FIG. 21). Then, using a BNC-to-wire cable, connect the BNC end to the “FLOW OUTPUT” on the flow meter's front panel. Connect the opposite wire ends of this cable to the Sparkfun Redboard-red wire to an analog input pin A0 of Redboard, and black wire to one of the ground (GND) pins on the Redboard (FIG. 21).

Compile and upload the Arduino script named “PID_flow_control.ino” (Table 1) to a Redboard. This script demonstrates servo control of the pump flow using a proportional-integral-derivative control. To define a setpoint flow for the servo control, be sure to refer to your Transonic flow probe to correctly convert between flow and the corresponding voltage signals. For the ME9PXL flow probe used in this study, there is a scaling factor of 100 to convert from flow rate in L/min to the corresponding digital value between 0 and 1023 read by the Redboard (e.g., 3 L/min is read as a digital value of 300). Use this scaling factor to define the value for the variable Setpointin the Arduino script, which corresponds to the proportional integral derivative (PID) controller's setpoint. The gain constants for proportional (variable is named Kp in the Arduino script), integral (Ki), and derivative (Kd) terms can also be adjusted to optimize for stability and oscillation. The provided script uses default values of 0.2, 0.5, and 0.1, respectively.

### Validation and Characterization:

Pressure-Flow Curves: Centrifugal pumps are characterized by the level of pressure and flow they can generate to achieve the hydraulic requirement. This relationship is visualized as pressure-flow (HQ) curves at varying speeds or voltage input levels. The hydraulic requirements of adult ECMO can vary depending on patient size and disease indication but typically operate at a pressure head between 250 and 300 mmHg and flow rate between 4 and 6 L/min. The HQ curves were generated for LivaNova Revolution, Medtronic Affinity, and Spectrum Quantum pump heads using the described flow loop circuit (Table 3, FIG. 20) with a 40% glycerin-saline mixture to match the viscosity of whole blood. The pump was operated between 6 and 12 volts at a 1-volt increment. Pressure head, circuit flow, rotational speed, and motor current and voltage were acquired to quantitatively evaluate pump performance. The pressure head, used herein, is defined as a pressure difference between the outlet and the inlet of the pump. An adjustable Hoffman clamp was used to control pump afterload to acquire an HQ curve for each input voltage level.

Hemolysis: Blood pumps can cause mechanical shear damage to cells over time, leading to hemolysis. To assess whether the pump console here causes any excessive unexpected levels of hemolysis, the pump console was evaluated over a 6-hour study at constant clinical pressure and flow conditions. The standard testing guideline by ASTM F1841-19: Standard Practice for Assessment of Hemolysis in Continuous Flow Blood Pumps was used. Bovine blood was procured from a local slaughterhouse (Anderson Meat and Processing) and heparinized at a concentration of 4000-6000 units/liter.

After assembling the flow loop circuit as detailed in FIG. 20 and Table 3, the circuit was initially primed with 200 mL of physiologic saline and circulated for at least 5 minutes to wet all blood-contacting surfaces. The flow loop circuit was then drained of saline completely and primed with 200 mL of bovine blood. The pump afterload and motor voltage were adjusted to achieve of 4.2 L/min of flow and 250-300 mmHg of pump pressure head. The circuit was run at the target setting for 5 minutes to equilibrate, and then a 6-hour test was started at this time of t=0 hours.

Every two hours, 1 mL of blood was also drawn to measure glucose, hematocrit, pH, and hemoglobin using a point-of-care blood gas analyzer. Every hour, a 2-mL blood sample was drawn from the circuit to measure plasma-free hemoglobin following Cripps' method. For this method, blood samples were first centrifuged at 1500 g for 20 minutes. The plasma's absorbance profile at light wavelengths of 576.5 nm (A_{576.5}), 560 nm (A₅₆₀), and 593 nm (A₅₉₃) were measured using SynergyHTX by BioTek, and these values were used to calculate plasma-free hemoglobin, in mg/dL, using the following formula:

$$177.6 \times ( {A}_{576.5} - {A}_{560} + {A}_{593} ) / 2$$

Then, the normalized index of hemolysis (NIH) was calculated using the following equation:

$$NIH ( g / 100 L ) = Δ freeHb \times V \times 100 - Ht / 100 \times 1 / Q \times T$$

where ΔfreeHb is the change in plasma free hemoglobin concentration (converted to g/L) relative to t=0 hour time point, V is the circuit blood volume in mL, Q is the blood flow rate in L/minute, Ht is the blood hematocrit, and Tis the time elapsed in minutes.

To verify that there is no background hemolysis under a static condition, a parallel static control testing was run by priming a bag with the same volume of blood as the pump testing and assessing for hemolysis and blood gas profile at the same specified time points.

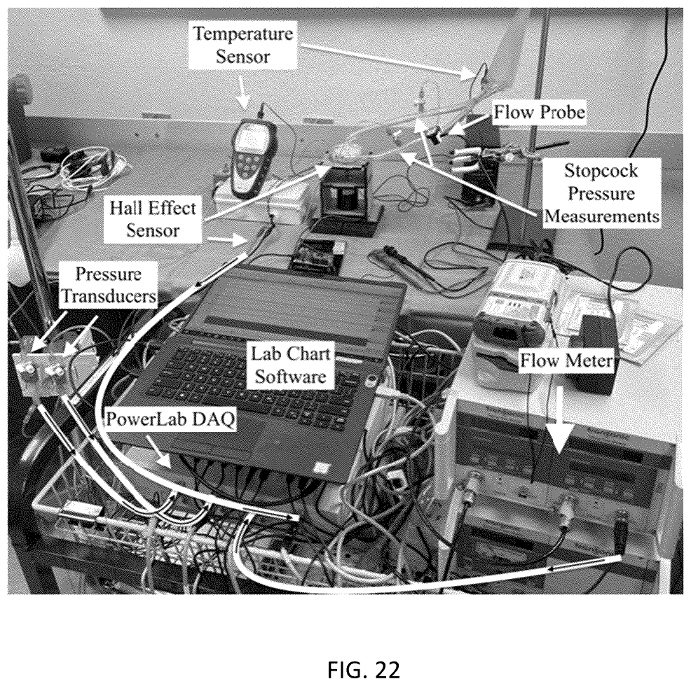

Data Monitoring: Table 4 summarizes the data acquisition setup used to generate the validation data. FIG. 22 shows the data acquisition setup during hemolysis testing. Detailed instructions on assembling a similar data acquisition setup and calibrating the sensors can also be found in a prior publication. Pump pressure head, Hall effect sensor and the corresponding motor/pump speed, and flow signals were all processed and recorded using the PowerLab and LabChart data acquisition system at a sampling frequency of 20,000 Hz. Individual fluid-filled pressure transducers were used to measure pump inlet and outlet pressures separately. Bridge amp front-end interface for PowerLab was used to receive the pressure transducer signals using Fogg System's interface cables. Male-to-male BNC cables were then used to connect from the bridge amp front end's output to the PowerLab's analog input. For pressure transducers, a two-point calibration at 0 and 80 mmHg was manually performed using a sphygmomanometer. Flow was measured using a tubing clamp-on probe and a flow meter discussed earlier. A male-male BNC cable was used to connect from the Transonic flow meter's flow output to an analog input channel of PowerLab. The flow probe was then calibrated in the LabChart software following the manufacturer's instructions for a two-point calibration between zero and full volt. Temperature was measured using a luer connector sensor and a temperature monitor every hour during hemolysis testing. The motor voltage and current were recorded from the DC variable power supply every hour during hemolysis testing. Pump speed was measured using a Hall effect sensor detailed earlier (FIG. 16). The Hall effect sensor's output and ground wires were connected to the alligator clip end of the alligator-to-male BNC connector cable and fed to one of the PowerLab analog input channels using the BNC end. The raw output signal from the Hall effect sensor is a square pulse wave. For the presented magnetic coupling disc, four square pulses from the Hall effect sensor correspond to one full rotation of the disc. This raw square wave signal can be converted to rotations per minute by dividing the measured frequency of this square wave by 4 and multiplying by 60.

TABLE 4
Summary of data acquisition equipment and sensors for
pressure-flow curve and hemolysis tests.

| | | | Source of | Material |
|---|---|---|---|---|
| Designator | Component | Quantity | Materials | Type |
| Electronic | 200 Ohm Resistor, | 1 Kit | Amazon | NA |
| | Jumper Wires Kit | | | |
| Enclosure | Hall Effect Sensor | 1 | DigiKey | NA |
| | 55100-3M-02-A | | | |
| Data | Flow Tubing | 1 | Transonic | NA |
| Acquisition | Clamp-On Probe | | | |
| | ME9PXL | | | |
| Data | Flow Meter | 1 | Transonic | NA |
| Acquisition | TS410 | | | |
| Data | Fluid Filled Pressure | 2 | Edwards | NA |
| Acquisition | Transducer | | Lifesciences | |
| | TruWave | | | |
| Data | Temperature Luer | 1 | PendoTech | NA |
| Acquisition | Connector Sensor | | | |
| | TM-TEMP-340 | | | |
| Data | Data Acquisition and | 1 | ADInstruments | NA |
| Acquisition | Analysis System | | | |
| | PowerLab | | | |
| Data | Bridge Amplifier | 1 | ADInstruments | |
| Acquisition | Quad FE224 | | | |
| Data | Interface cable | 2 | Fogg System | |
| Acquisition | between | | | |
| | TruWave Edwards | | | |
| | Lifesciences and | | | |
| | Bridge Amp | | | |
| | Catalog # 0395-2521 | | | |
| Data | Arterial Blood Gas | 1 | Heska | NA |
| Acquisition | Machine ePOC | | | |
| Data | Male-Male BNC | 1 | Digi-Key | NA |
| Acquisition | coaxial cable | | | |
| | 3272-CO- | | | |
| | 058BNCX200- | | | |
| | 004-ND | | | |
| Data | Alligator clip-to-Male | 1 | Digi-Key | NA |
| Acquisition | BNC connector cable | | | |
| | 1286-1232-ND | | | |
| Data | BNC-to-wire cable | 1 | Digi-Key | NA |
| Acquisition | 501-1031-ND | | | |

HQ Curves: The LivaNova Revolution pump achieved pump speeds between 1300 to 3000 RPM for voltage inputs between 6 and 12 volts, as shown in FIG. 5 (top panel). Slight variations in the RPM for a fixed input voltage were due to variations in the pump afterload, where higher pressure head/lower flow correlated with faster pump speed, and lower pressure/faster flow correlated with lower pump speed. At 12 volts, the pump met the clinically relevant levels of flow and pressure for extracorporeal applications at >250 mmHg and >4 L/min, as shown in FIG. 5 (bottom panel). Two other clinical centrifugal pumps-Medtronic Affinity and Spectrum Medical Quantum CP-were tested but did not attain as high of pressure or RPM as the LivaNova Revolution (see the Zenodo repository for their HQ curves).

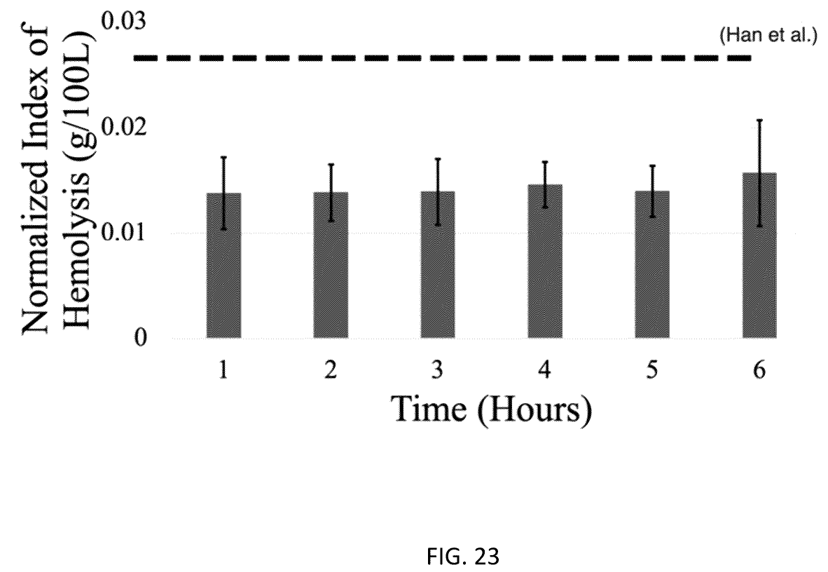

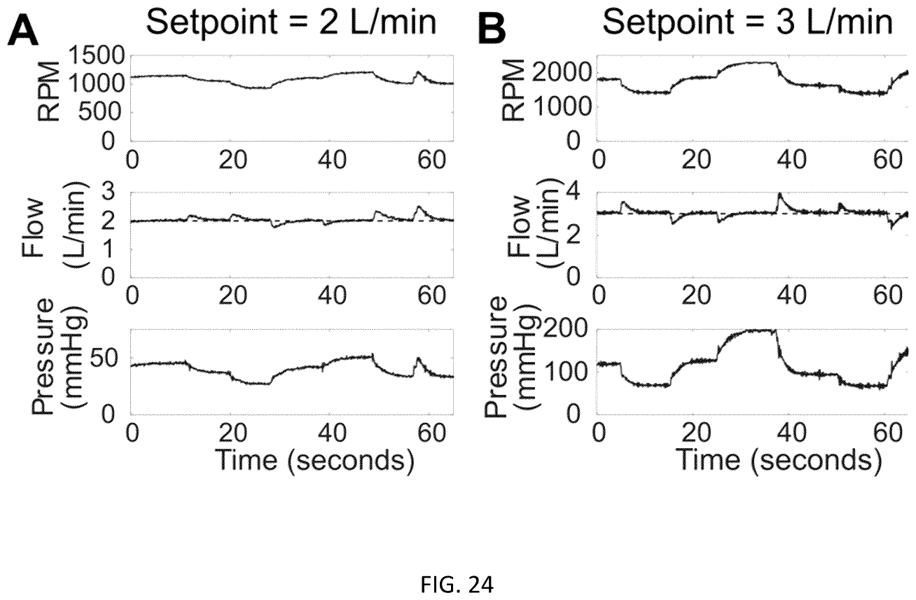

Hemolysis: The Smart Blood Pump console was assessed for pump biocompatibility, again using the LivaNova pump head as it achieved the most clinically relevant levels of flow and pressure. The acquired blood showed normal values prior to testing: the average hematocrit was 35±2%, glucose was 117±9 mg/dL, and pH was 7.31±0.04 (n=3). Average flow, pressure, hematocrit, temperature, and current during the 6-hour test are summarized in Table 5. The static control did not show any appreciable change in plasma-free hemoglobin over time. At a 6-hour mark, which serves as a benchmark for blood pumps, the normalized index of hemolysis was 0.014±0.0028 grams hemoglobin/100 L blood (FIG. 23, n=3). This value was a lower hemolytic index than a previously reported value that was calculated for a comparable but slightly higher flow and pressure profile (0.0287±0.0041 g/100 L). Therefore, our console shows comparable biocompatibility to that of a clinically approved console.

TABLE 5
Data summary from the six-hour hemolysis testing presented
as mean ± standard deviation (N = 3)

| | Hour 0 | Hour 2 | Hour 4 | Hour 6 |
|---|---|---|---|---|
| Hematocrit (%) | 35 ± 2 | 33 ± 1 | 32 ± 1 | 31 ± 2 |
| Glucose (mg/dL) | 114 ± 5 | 81.3 ± 16.3 | 62 ± 17.5 | 45 ± 16.7 |
| Temperature (C.) | 25.18 ± 0.97 | 37.67 ± 1.10 | 37.68 ± 0.96 | 37.53 ± 1.05 |
| Motor Current (A) | 1.7 | 1.7 | 1.7 | 1.7 |
| Input Voltage (V) | 12 | 12 | 12 | 12 |
| Flow (L/min) | 4.16 ± 0.1 | 4.16 ± 0.1 | 4.11 ± 0.05 | 4.09 ± 0.1 |
| RPM | 2776 ± 43 | 2782 ± 41 | 2773 ± 39 | 2776 ± 16 |
| Pressure Head | 274 ± 9 | 280 ± 14 | 281 ± 12 | 281 ± 14 |
| (mmHg) | | | | |

Pulsatility of LivaNova Revolution: Pulsatile speed, flow, and pressure were generated using LivaNova circuit and 40% glycerol solution. Representative waveforms in FIG. 6 demonstrate oscillation between 2200 and 2800 RPM, 170 and 300 mmHg, and 4 and 5 L/min. These pulsatile profiles were achieved by adjusting the variables in the Arduino script titled “pulsatile_flow_control.ino”: minPWM and maxPWM of 180 and 255, respectively, and period of 1000 milliseconds.

Servo Flow Control: FIG. 24 demonstrates that the Smart Blood Pump can be servo controlled using PID algorithm to maintain target flow. The data were generated using Kp, Ki, and Kd values of 0.2, 0.5, and 0.1, respectively, in the Arduino script titled “PID_flow_control.ino.” The setpoint flow rate is shown as a dotted line for setpoint of 2 L/min in FIG. 18A and 3 L/min in FIG. 24 (panel B). The flow disturbances, caused by acute occlusion or release of the Hoffman clamp, are quickly corrected.

Future Directions: Future console design needs to expand its compatibility with other clinical pump head types to serve as a more universal platform, which is a lacking feature in many pump consoles in general but demonstrated by Anivia Extracorporeal Support System. The presented console and enclosure were most compatible with the LivaNova Revolution, and much less so with Medtronic Affinity or Spectrum Medical Quantum. The Medtronic Affinity pump has a thicker casing at the bottom which likely reduced torque transmission from the magnetic disc to its impellers. The Spectrum Medical Quantum pump's impeller diameter is smaller than either Medtronic Affinity or LivaNova Revolution. Due to its specific customization for the LivaNova Revolution, the fit was suboptimal for these other two pump head types. Further optimization and customization of our enclosure likely improve the performance with these other pumps. For example, a more powerful motor or magnetic coupling disc can be used to overcome the larger separation distance. Another future direction is to use the Smart Blood Pump console to implement more advanced forms of feedback control using physiologic sensors compared to simply keeping a constant flow as demonstrated here. Designing a safe automation strategy for extracorporeal life support requires a programmable blood pump that can be tuned to optimize for stability and control, so this investigation is a logical extension of the presented work here.

In conclusion, this invention discloses a rapidly available, inexpensive console to operate centrifugal blood pumps for extracorporeal life support applications. This technology will foster research and innovation in this scientific and clinical area to ultimately develop a smarter blood pump.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### LIST OF REFERENCES

[1]. ELSO International Summary of Statistics|ECMO|ECLS (2024) (n.d.). https://www.elso.org/registry/internationalsummaryandreports/internationalsummary.aspx
[2]. Schmidt M, Hajage D, Lebreton G, Monsel A, Voiriot G, Levy D, Baron E, Beurton A, Chommeloux J, Meng P, Nemlaghi S, Bay P, Leprince P, Demoule A, Guidet B, Constantin J M, Fartoukh M, Dres M, Combes A, Luyt C-E, Hekimian G, Brechot N, de Chambrun M P, Desnos C, Arzoine J, Guerin E, Schoell T, Demondion P, Juvin C, Nardonne N, Marin S, D'Alessandro C, Nguyen B-L, Quemeneur C, James A, Assefi M, Lepere V, Savary G, Gibelin A, Turpin M, Elabbadi A, Berti E, Vezinet C, Bonvallot H, Delmotte P-R, Sarcus M D, Tour C D F D L, Abbas S, Maury E, Baudel J-L, Lavillegrand J-R, Oufella H A, Abdelkrim A, Urbina T, Virolle S, Deleris R, Bonny V, Marec J L, Mayaux J, Morawiec E (2020) Extracorporeal membrane oxygenation for severe acute respiratory distress syndrome associated with COVID-19: a retrospective cohort study. Lancet Respir Med 8:1121-1131. https://doi.org/10.1016/S2213-2600 (20) 30328-3.
[3]. Gannon W D, Stokes J W, Francois S A, Patel Y J, Pugh M E, Benson C, Rice T W, Bacchetta M, Semler M W, Casey J D Association between Availability of Extracorporeal Membrane Oxygenation and Mortality in Patients with COVID-19 Eligible for Extracorporeal Membrane Oxygenation: A Natural Experiment, Am. J. Respir. Crit. Care Med. 205 (n.d.) 1354-1357. https://doi.org/10.1164/rccm.202110-2399LE
[4]. Oude Lansink-Hartgring A, van Minnen O, Vermeulen K M, van den Bergh W M, Dutch Extracorporeal Life Support Study Group (2021) Hospital Costs of Extracorporeal Membrane Oxygenation in Adults: A Systematic Review. PharmacoEconomics-Open 5:613-623. https://doi.org/10.1007/s41669-021-00272-9
[5]. Mishra V, Svennevig J L, Bugge J F, Andresen S, Mathisen A, Karlsen H, Khushi I, Hagen T P (2010) Cost of extracorporeal membrane oxygenation: evidence from the Rikshospitalet University Hospital, Oslo, Norway. Eur J Cardiothorac Surg 37:339-342. https://doi.org/10.1016/j.ejcts.2009.06.059
[6]. Wang S, Moroi M K, Kunselman A R, Myers J L, Ündar A (2020) Evaluation of centrifugal blood pumps in term of hemodynamic performance using simulated neonatal and pediatric ECMO circuits. Artif Organs 44:16-27. https://doi.org/10.1111/aor.13436
[7]. Harvard Apparatus C, Pump (2024) accessed July 5, (n.d.). https://www.harvardapparatus.com/catalog/product/view/id/8293/s/centrifugal-pump/category/522/[8].
[7]. Schraven L, Kaesler A, Flege C, Kopp R, Schmitz-Rode T, Steinseifer U, Arens J (2018) Effects of Pulsatile Blood Flow on Oxygenator Performance. Artif Organs 42:410-419. https://doi.org/10.1111/aor.13088
[9]. Sakurai H, Fujiwara T, Ohuchi K, Hijikata W, Inoue Y, Maruyama O, Tahara T, Yokota S, Tanaka Y, Takewa Y, Mizuno T, Arai H (2023) Innovative experimental animal models for real-time comparison of antithrombogenicity between two oxygenators using dual extracorporeal circulation circuits and indocyanine green fluorescence imaging. Artif Organs 47:77-87. https://doi.org/10.1111/aor.14380
[10]. Woelke E, Mager I, Schmitz-Rode T, Steinseifer U, Clauser J C (2021) Ann Biomed Eng 49:3165-3175. https://doi.org/10.1007/s10439-021-02849-1. Validation of a Miniaturized Test Loop for the Assessment of Human Blood Damage by Continuous-Flow Left-Ventricular Assist Devices
[11]. Woelke E, Klein M, Mager I, Schmitz-Rode T, Steinseifer U, Arens J, Clauser J C (2020) Ann Biomed Eng 48:768-779. https://doi.org/10.1007/s10439-019-02404-z. Miniaturized Test Loop for the Assessment of Blood Damage by Continuous-Flow Left-Ventricular Assist Devices
[12]. Tonna J E, Abrams D, Brodie D, Greenwood J C, Mateo-Sidron JARUBIO, Usman A, Fan E (2021) Management of Adult Patients Supported with Venovenous Extracorporeal Membrane Oxygenation (VV ECMO): Guideline from the Extracorporeal Life Support Organization (ELSO). ASAIO J 67:601. https://doi.org/10.1097/MAT.0000000000001432
[13]. Gajkowski E F, Herrera G, Hatton L, Velia Antonini M, Vercaemst L, Cooley E (2022) ELSO Guidelines for Adult and Pediatric Extracorporeal Membrane Oxygenation Circuits. ASAIO J 68:133. https://doi.org/10.1097/MAT.0000000000001630.
[14]. F04 Committee, Practice for Assessment of Hemolysis in Continuous Flow Blood Pumps, International ASTM n.d. https://doi.org/10.1520/F1841-19.
[15]. Cripps C M (1968) Rapid method for the estimation of plasma haemoglobin levels. J Clin Pathol 21:110-112. https://doi.org/10.1136/jcp.21.1.110
[16]. Malinauskas R A (1997) Plasma Hemoglobin Measurement Techniques for the In Vitro Evaluation of Blood Damage Caused by Medical Devices. Artif Organs 21:1255-1267. https://doi.org/10.1111/j.1525-1594.1997.tb00486.x
[17]. Naito K, Mizuguchi K, Nose Y (1994) The need for standardizing the index of hemolysis. Artif Organs 18:7-10. https://doi.org/10.1111/j.1525-1594.1994.tb03292.x
[18]. Ukita R, Wu W K, Liang J, Talackine J R, Patel Y J, Francois S A, Cardwell N L, Flynn C R, Shingina A, Washington M K, Trinh V Q-H, Bacchetta M, Alexopoulos S P (2023) Pulmonary artery banding in sheep: a novel large animal model for congestive hepatopathy. J Appl Physiol 134:840-845. https://doi.org/10.1152/japplphysiol.00473.2022
[19]. Han D, Leibowitz J L, Han L, Wang S, He G, Griffith B P, Wu Z J (2022) Computational fluid dynamics analysis and experimental hemolytic performance of three clinical centrifugal blood pumps: Revolution, Rotaflow and CentriMag, Med. Nov Technol Devices 15:100153. https://doi.org/10.1016/j.medntd.2022.100153.
[20]. Vercaemst L (2024) Year in review: Highlights in ECLS Innovation and Technology, Anno 2022-2023. Perfusion 39:31-35. https://doi.org/10.1177/02676591231195691
[21]. ANIVIA SG1000 Pump Console (2024) (n.d.). https://www.apmtd.com/products-solutions/.
[22]. Conway R G, Berk Z B, Zhang J, Li T, Tran D, Wu Z J, Griffith B P (2020) Evaluation of an autoregulatory ECMO system for total respiratory support in an acute ovine model. Artif Organs 44:478-487. https://doi.org/10.1111/aor.13618.
[23]. Spencer B L, Shaikh N, Gudex L, Dann T, Langley M, Matich H, Bartlett R H, Rojas-Peña A, Potkay J A (2023) In Vivo Testing of an Ambient Air Based, Portable, and Automated CO2 Removal Controller for Artificial Lungs. ASAIO J 69. e301. https://doi.org/10.1097/MAT.0000000000001968.
[24]. Conway, R. Gregory, Bartley Griffith, and Zhongjun Wu. System for control of a blood gas exchanger. World Intellectual Property Organization WO2019246320A1, filed Jun. 20, 2019, and issued Dec. 26, 2019. https://patents.google.com/patent/WO2019246320A1/en?inventor=bartley+griffith&oq=bartley+griffith.
[25]. Mechoor, Rahul Raj, Tyler Schmidt, and Ethan Kung. “A Real-Time Programmable Pulsatile Flow Pump for In Vitro Cardiovascular Experimentation.” Journal of Biomechanical Engineering 138, no. 11 (Nov. 1, 2016): 111002. https://doi.org/10.1115/1.4034561.
[26]. Göbel, C, A Arvand, G Rau, H Reul, B Meyns, W Flameng, R Eilers, and O Marseille. “A New Rotary Blood Pump for Versatile Extracorporeal Circulation: The DeltaStream™.” Perfusion 17, no. 5 (September 2002): 373-82. https://doi.org/10.1191/0267659102pf602oa.
[27]. Patel, Krishna, and Akif Ündar. “Centrifugal Pump Generates Superior Hemodynamic Performance Compared to a New Diagonal Blood Pump in Neonatal and Pediatric ECMO Circuits.” World Journal for Pediatric and Congenital Heart Surgery 13, no. 2 (March 2022): 235-41. https://doi.org/10.1177/21501351211057426.

What is claimed is:

1. A blood pump system, comprising:
a blood pump configured to provide a blood flow in an extracorporeal membrane oxygenation (ECMO) circuit for extracorporeal physiological support for a patient;
a magnetic coupling member configured to drive the blood pump to operably provide the blood flow;
a direct current (DC) motor configured to drive the magnetic coupling member, thereby driving the blood pump; and
a controller configured to regulate a motor speed of the DC motor.

2. The blood pump system of claim 1, wherein the blood pump is a standard ECMO blood pump, or a customized blood pump.

3. The blood pump system of claim 1, wherein the blood flow is a constant blood flow, or a pulsatile blood flow.

4. The blood pump system of claim 1, wherein the magnetic coupling member is configured to rotate a pump impeller of the blood pump in a contactless manner.

5. The blood pump system of claim 1, further comprising:
a Hall effect sensor positioned in relation to the magnetic coupling member to measure a pump speed of the blood pump in real time,
wherein the controller is further configured to receive a signal of the pump speed from the Hall effect sensor and regulate the motor speed of the DC motor responsively.

6. The blood pump system of claim 1, further comprising:
a physiological sensor disposed in the ECMO circuit for monitor physiological parameters of the patient.

7. The blood pump system of claim 6, wherein the controller is further configured to receive a physiological input from the physiological sensor in the ECMO circuit and regulate the motor speed responsively, thereby adjusting the pump speed in a closed-loop feedback system.

8. The blood pump system of claim 1, wherein the controller is further configured to receive a signal from the patient about its vital state and regulate the motor speed responsively, thereby adjusting the pump speed.

9. The blood pump system of claim 1, wherein the controller is further configured to receive a user-scripted input to operate the blood pump system in a pulsatile or periodic manner.

10. The blood pump system of claim 1, wherein the blood pump system is programed to vary the motor speed in a constant manner or a pulsatile manner.

11. The blood pump system of claim 1, further comprising:
a feedback mechanism for controlling the pump speed by using venous saturation as a surrogate measure for patient status and basis for speed adjustment, so as to enable automation according to metabolic need of the patient.

12. The blood pump system of claim 11, wherein the feedback mechanism comprises an oxygen sensor coupled in the ECMO circuit to monitor an oxygen level, an oxygen consumption, and/or oxygen saturation (SvO₂) of the patient.

13. The blood pump system of claim 11, wherein the blood pump system is programmed, such that the pump speed is automatically adjusted to maintain a setpoint flow for physiologic oxygen saturation.

14. The blood pump system of claim 1, further comprising:
a safety mechanism for ensuring the blood pump not to blindly increase speed in response to an abnormal ECMO circuit condition including circuit obstruction, low fluid volume status, clot formation, a suction event, pump thrombosis, and/or circuit thrombosis.

15. The blood pump system of claim 1, being operable at clinically relevant levels of flow and pressure needed in extracorporeal life support applications.

16. A method of operating the blood pump system of claim 1 for extracorporeal life support applications, comprising:
driving the DC motor, thereby the blood pump at a target pump speed to provide the blood flow in the ECMO circuit for extracorporeal physiological support for the patient in an automatic mode or a manual mode, wherein the target pump speed is determined based on preset operation parameters.

17. The method of claim 16, wherein the preset operation parameters include setpoint flow and/or pressure in the ECMO circuit, motor current, oxygen saturation, and/or a physiological state of the patient.

18. The method of claim 17, wherein the physiological state of the patient includes an oxygen level, an oxygen consumption and/or oxygen saturation.

19. The method of claim 16, further comprising:
measuring the pump speed of in real time; and
regulating the motor speed responsively to adjust the pump speed when the measured pump speed is deviated from the target pump speed.

20. The method of claim 16, further comprising:
measuring the SvO₂; and
regulating the motor speed responsively to adjust the pump speed when the measured SvO₂ is deviated from a setpoint value.

21. The method of claim 16, further comprising:
measuring the flow rate and/or pressure in the ECMO circuit; and
regulating the motor speed responsively to adjust the pump speed when the measured flow rate and/or pressure are deviated from the setpoint flow and/or pressure.

22. The method of claim 16, further comprising:
detecting an abnormal ECMO circuit condition including circuit obstruction, low fluid volume status, clot formation, a suction event, pump thrombosis, and/or circuit thrombosis; and
regulating the motor speed responsively to ensure the blood pump not to blindly increase speed when the abnormal ECMO circuit condition is detected.

23. The method of claim 22, wherein when the abnormal ECMO circuit condition is detected, the blood pump system exits from the automatic mode and enters the manual mode until the abnormal ECMO circuit condition is resolved.
